(19) United States
(12) Patent Application Publication
(10) Pub. No.: US 2025/0069709 A1
(43) Pub. Date: Feb. 27, 2025

(54) METHOD AND APPARATUS FOR TRAINING MOLECULAR GENERATIVE MODEL, DEVICE, AND STORAGE MEDIUM

(71) Applicant: Tencent Technology (Shenzhen) Company Limited, Shenzhen (CN)

(72) Inventors: Jonathan Pradana MAILOA, Shenzhen (CN); Jiezhong Qiu, Shenzhen (CN); Shengyu Zhang, Shenzhen (CN)

(73) Assignee: Tencent Technology (Shenzhen) Company Limited, Shenzhen (CN)

(21) Appl. No.: 18/943,413

(22) Filed: Nov. 11, 2024

Related U.S. Application Data
(63) Continuation of application No. PCT/CN2023/097469, filed on May 31, 2023.

(30) Foreign Application Priority Data
Dec. 15, 2022 (CN) 202211612755.8

(51) Int. Cl. G16C 20/70; G16C 20/30

(52) U.S. Cl. CPC G16C 20/70; G16C 20/30

(57) ABSTRACT

This application relates to quantum chemistry. The method includes: obtaining training data for a molecular generative model; predicting, if a labeled property value in molecular property label data of a sample molecule in the training data for at least one of M properties is missing, a property value of the sample molecule for at least one property, to obtain molecular property prediction data of the sample molecule; obtaining molecular property tag data of the sample molecule based on the molecular property label data and the molecular property prediction data of the sample molecule; and training the molecular generative model based on the molecular property tag data of the sample molecule, to obtain a trained molecular generative model. This application supports training of the molecular generative model by using the training data without the labeled property value, molecular properties are more abundant and diversity of molecular data is improved.

### RELATED APPLICATION

This application is a continuation application of PCT Patent Application No. PCT/CN2023/097469, filed with the China National Intellectual Property Administration, PRC on May 31, 2023, which claims priority to Chinese Patent Application No. 202211612755.8, filed with the China National Intellectual Property Administration, PRC on Dec. 15, 2022, each of which is incorporated herein by reference in its entirety.

### FIELD OF THE TECHNOLOGY

Embodiments of this disclosure relate to the field of quantum chemistry, and in particular, to a method and apparatus for training a molecular generative model, a device, and a storage medium.

### BACKGROUND OF THE DISCLOSURE

In the field of quantum chemistry, a new molecule is generated through a molecular generative model, so as to greatly reduce generation costs of the new molecule.

In the related art, the molecular generative model is usually trained by using complete labeled data, so that a trained molecular generative model may be configured for generating a new molecule. The new molecule is related to a property of a molecule in the complete labeled data. For any molecule, any property corresponding to the molecule has a labeled property value in the complete labeled data.

However, there are relatively few property types of the molecules in the complete labeled data. The molecular generative model trained based on the complete labeled data also has fewer property options for generating new molecules, resulting in fewer types of the new molecules that can be generated by the molecular generative model.

### SUMMARY

Embodiments of this disclosure provide a method and apparatus for training a molecular generative model, a device, and a storage medium. The technical solutions are as follows.

According to an aspect of the Embodiments of this disclosure, a method for training a molecular generative model is provided, the method being performed by a computer device, and including:
obtaining training data for a molecular generative model, the training data including molecular property label data of each of a plurality of sample molecules, each piece of molecular property label data including a labeled property value of the sample molecule for M properties, M being a positive integer;
determining, if the labeled property value in the molecular property label data of a sample molecule in the training data for at least one of the M properties is missing, that the molecular property label data of the sample molecule is incomplete, and predicting a property value of the sample molecule for the at least one property, to obtain molecular property prediction data of the sample molecule;
obtaining molecular property tag data of the sample molecule based on the molecular property label data and the molecular property prediction data of the sample molecule; and
training the molecular generative model based on the molecular property tag data of the sample molecule, to obtain a trained molecular generative model.

According to an aspect of the Embodiments of this disclosure, an apparatus for training a molecular generative model is provided, the apparatus including:
a data obtaining module, configured to obtain training data for a molecular generative model, the training data including molecular property label data of each of a plurality of sample molecules, each piece of molecular property label data including a labeled property value of the sample molecule for M properties, M being a positive integer;
a data prediction module, configured to determine, if the labeled property value in the molecular property label data of a sample molecule in the training data for at least one of the M properties is missing, that the molecular property label data of the sample molecule is incomplete, and predict a property value of the sample molecule for the at least one property, to obtain molecular property prediction data of the sample molecule;
a tag obtaining module, configured to obtain molecular property tag data of the sample molecule based on the molecular property label data and the molecular property prediction data of the sample molecule; and
a model training module, configured to train the molecular generative model based on the molecular property tag data of the sample molecule, to obtain a trained molecular generative model.

According to an aspect of the Embodiments of this disclosure, a computer device is provided, including a processor and a memory, the memory having a computer program stored therein, the computer program being loaded and executed by the processor to implement the foregoing method.

According to an aspect of the Embodiments of this disclosure, a non-transitory computer-readable storage medium is provided, having a computer program stored therein, the computer program being loaded and executed by a processor to implement the foregoing method.

According to an aspect of the Embodiments of this disclosure, a computer program product is provided, the computer program product including a computer program, the computer program being stored in a non-transitory computer-readable storage medium. A processor of a computer device reads the computer program from the computer-readable storage medium, and the processor executes the computer program, so that the computer device performs the foregoing method.

The technical solutions provided in the Embodiments of this disclosure may include the following beneficial effects.

The molecular generative model is trained by using the training data with a missing labeled property value, so that newly generated molecular data has more abundant molecular properties that are not limited to a few molecular properties specified in complete labeled data, thereby improving diversity of the generated molecular data. In addition, the training data may include a plurality of pieces of complete labeled data. Therefore, types of the molecular properties of the sample molecule can be enriched through the training data, thereby further improving diversity of output results of the molecular generative model.

In addition, since the labeled property values for the M properties in the training data are missing, a data gap in incomplete labeled data is filled in by obtaining the molecular property prediction data corresponding to the sample molecule, so as to improve completeness of the training sample, achieve a smoother subsequent training process, and facilitate improvement in training accuracy of the molecular generative model.

In addition, different from a training manner in which the molecular generative model is trained by using only the complete labeled data in the related art, in the technical solutions provided in the Embodiments of this disclosure, the training data with a missing labeled property value may also be configured for training the molecular generative model. Therefore, the method for training a molecular generative model is also enriched.

### BRIEF DESCRIPTION OF THE DRAWINGS

FIG. 1 is a schematic diagram of an implementation environment according to an embodiment of this disclosure.

FIG. 2 is a schematic diagram of a method for training a molecular generative model according to an embodiment of this disclosure.

FIG. 3 is a schematic diagram of a method for training a semi-supervised sequential variational autoencoder (SSVAE) model in the related art.

FIG. 4 is a schematic diagram of another method for training an SSVAE model in the related art.

FIG. 5 is a schematic diagram of a data merging manner in the related art.

FIG. 6 is a flowchart of a method for training a molecular generative model according to an embodiment of this disclosure.

FIG. 7 is a flowchart of a method for training a molecular generative model according to another embodiment of this disclosure.

FIG. 8 is a schematic diagram of a mask matrix according to an embodiment of this disclosure.

FIG. 9 is a schematic diagram of a method for training a ConGen model (a molecular generative model) according to an embodiment of this disclosure.

FIG. 10 is a flowchart of a method for training a molecular generative model according to another embodiment of this disclosure.

FIG. 11 is a flowchart of a method for training a molecular generative model according to another embodiment of this disclosure.

FIG. 12 is a schematic diagram of a method for training a molecular generative model and a molecular property prediction model according to an embodiment of this disclosure.

FIG. 13 is a schematic diagram of performance comparison of a model after being switched from TensorFlow 1.0 to a PyTorch platform according to an embodiment of this disclosure.

FIG. 14 is a schematic diagram of a molecular generation method according to an embodiment of this disclosure.

FIG. 15 is a block diagram of an apparatus for training a molecular generative model according to an embodiment of this disclosure.

FIG. 16 is a block diagram of an apparatus for training a molecular generative model according to another embodiment of this disclosure.

FIG. 17 is a structural block diagram of a computer device according to an embodiment of this disclosure.

### DESCRIPTION OF EMBODIMENTS

To make objectives, technical solutions, and advantages of this application clearer, implementations of this application are to be further described in detail below with reference to the accompanying drawings.

Before the technical solutions of this application are described, some background technology knowledge involved in this application is described first. As an optional solution, the following related technologies may be arbitrarily combined with the technical solutions of the Embodiments of this disclosure, and all fall within the protection scope of the Embodiments of this disclosure. The Embodiments of this disclosure include at least part of the following content.

Artificial intelligence (AI) is a theory, a method, a technology, and an application system that use a digital computer or a machine controlled by the digital computer to simulate, extend, and expand human intelligence to sense an environment, obtain knowledge, and obtain an optimal result with knowledge. In other words, AI is a comprehensive technology in computer science and attempts to understand the essence of intelligence and produce a new intelligent machine that can react in a manner similar to human intelligence. AI is to study the design principles and implementation methods of various intelligent machines, to enable the machines to have the functions of perception, reasoning, and decision-making.

The AI technology is a comprehensive discipline, and involves a wide range of fields including both hardware-level technologies and software-level technologies. The basic AI technologies generally include technologies such as a sensor, a dedicated AI chip, cloud computing, distributed storage, a big data processing technology, an operating/interaction system, and electromechanical integration. AI software technologies mainly include several major directions such as a natural language processing technology and machine learning/deep learning.

Machine learning (ML) is a multi-disciplinary interdiscipline, involving a plurality of disciplines such as the probability theory, statistics, approximation theory, convex analysis, and algorithm complexity theory. The ML specializes in how a computer simulates or realizes learning behaviors of human to obtain new knowledge or skills, and reorganizes relevant knowledge structures to keep improving performance thereof. ML is the core of AI and is a fundamental way to make computers intelligent, which is applied in all fields of AI. The ML and the deep learning generally include technologies such as an artificial neural network, a confidence network, reinforcement learning, transfer learning, inductive learning, and learning from demonstration.

Deep learning (DL) is a new research direction in the field of ML, which is introduced into ML to make DL closer to the original goal, i.e., AI. DL is to learn an internal law and a representation level of sample data, and the information obtained during the learning is of great help to interpretation of data such as a text, an image, and a sound. An ultimate goal of DL is to enable a machine to have analysis and learning capabilities like a person and recognize the data such as the text, the image, and the sound. DL is a complex ML algorithm that has achieved results in speech and image recognition that far exceed the previous related art. DL has achieved many results in search technology, data mining, ML, machine translation, natural language processing, multimedia learning, speech, recommendation and personalization technology, and other related fields. DL enables a machine to imitate human activities such as seeing, hearing, and thinking, thereby solving many complex pattern recognition problems, to enable the AI-related technologies to make great progress.

With the research and progress of AI technologies, the AI technology has been studied and applied in a plurality of fields such as a common smart home, a smart wearable device, a virtual assistant, a smart speaker, smart marketing, unmanned driving, automatic driving, an unmanned aerial vehicle, a robot, smart medical care, smart customer service, virtual reality (VR), augmented reality (AR), a game, virtual human, and digital human. It is believed that with the development of technologies, the AI technology is to be applied in more fields and plays increasingly important value.

The solutions provided in the Embodiments of this disclosure involve the technologies such as ML of AI, which are specifically described by using the following embodiments.

Before the technical solutions of this application are described, some terms involved in this application are explained first. As an optional solution, the following related explanations may be arbitrarily combined with the technical solutions of the Embodiments of this disclosure, and all fall within the protection scope of the Embodiments of this disclosure. The Embodiments of this disclosure include at least part of the following content.

Recurrent neural network (RNN): It is a special type of neural network structure, which is proposed based on a point of view that “human cognition is based on past experience and memory”. It not only considers an input at a previous moment, but also gives the network a “memory” function for previous content. A reason why the RNN is referred to as the recurrent neural network is that a current output of a sequence is also related to the previous output. A specific manifestation is that the network memorizes previous information and applies the previous information to calculation of the current output. To be specific, nodes between hidden layers are no longer disconnected but connected, and inputs of the hidden layers not only include an output of an input layer but also include an output of the hidden layer at a previous moment.

Variational autoencoder (VAE): A bunch of real samples are transformed into an ideal data distribution through an encoder network, and then the data distribution is delivered to a decoder network to obtain a bunch of generated samples. If the generated samples are close enough to the real samples, an autoencoder model is trained.

Semi-supervised sequential variational autoencoder (SSVAE): It is a baseline model compared with a ConGen model in the Embodiments of this disclosure. The SSVAE model relies on a variational autoencoding algorithm and a conditional input to implement a conditional molecular structure generation capability thereof. However, the baseline model SSVAE has significant practical limitations (ConGen has overcome these limitations).

ConGen: It is a conditional molecular generative model discussed in the Embodiments of this disclosure. It is a novel ML model based on an autoencoder that can generate a multi-conditional molecule even if training data used has a completely missing or incomplete property tag (i.e. a labeled property value).

PyTorch: It is a Python ML library, and is generally used for a neural network or another gradient-related algorithm.

TensorFlow: It is another Python ML library with almost the same function as PyTorch. However, a significant difference in design concept exists between TensorFlow and PyTorch. TensorFlow generally needs to define a model by using a static graph, precompile the static graph before running, and optimize efficiency based on the maximum runtime, and PyTorch has greater flexibility at runtime based on a dynamic graph, but sacrifices some computational efficiency.

Simplified molecular-input line-entry system (SMILES): This is a specification for describing a structure of a chemical substance using short ASCII strings. The SMILES strings are designed to be configured for directly representing a geometrical shape of a molecular chemical structure, so that the strings may be configured for representation of an encoder input and a decoder output of a generative model.

Positive semi-definite (PSD) matrix: It is a matrix M that satisfies a special condition, where for any column vector z of real numbers, a real number z^{T}Mz is positive or zero, and z^{T} is the transpose of z. All eigenvalues of the PSD matrix M are non-negative.

Predictor: It is a sub-model in the ConGen model responsible for prediction of molecular properties. An input of the predictor is a molecular structure (which may be represented by a SMILES string or another molecular structure representation scheme). In some embodiments, the property in the Embodiments of this disclosure refers to a property of a molecule, which may also be referred to as a molecular property.

Encoder: It is a sub-model in the ConGen model responsible for encoding an inputted high-dimensional structure into a low-dimensional latent space representation.

Decoder: It is a sub-model in the ConGen model responsible for decoding the low-dimensional latent space representation (from the encoder) and the molecular property (from the predictor or a training tag) back to an original molecular structure representation (namely, an input to the encoder).

Unconditional generation: When a molecule is generated by using the decoder in ConGen, any required property does not need to be specified for the generated molecule.

Conditional generation: When a molecule is generated by using the decoder in ConGen, one or more required properties are simultaneously specified for the generated molecule.

Covariance matrix: It is a correlation matrix among the molecular properties in the training data. For example, if a tag matrix of the training data of a size nₛ×nₚ (nₛ is a quantity of sample molecules in the training data, and nₚ is a quantity of molecular properties configured for training/generation) is given, a size of the covariance matrix is nₚ×nₚ.

In the method provided in the Embodiments of this disclosure, each operation may be performed by a computer device. The computer device refers to an electronic device having data computing, processing, and storage capabilities. The computer device may be a terminal such as a personal computer (PC), a tablet computer, a smartphone, a wearable device, or an intelligent robot, or may be a server. The server may be an independent physical server, or may be a server cluster composed of a plurality of physical servers or a distributed system, and may further be a cloud server providing a cloud computing service.

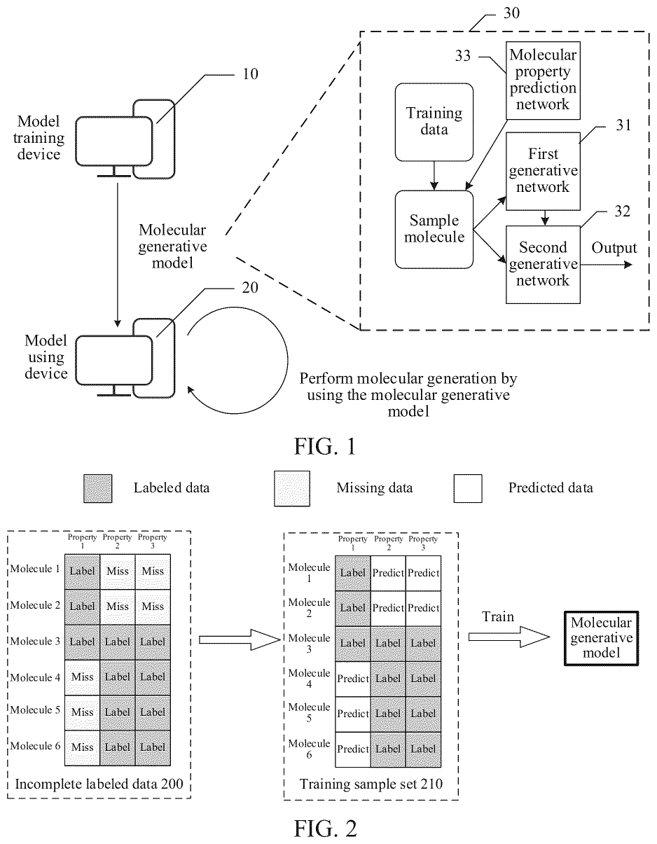

FIG. 1 is a schematic diagram showing an implementation environment according to an embodiment of this disclosure. The implementation environment may include a model training device 10 and a model using device 20.

The model training device 10 may be an electronic device such as a PC, a computer, a tablet computer, or an intelligent robot, or some other electronic devices having stronger computing power. The model training device 10 may further be a server. The model training device 10 is configured to train a molecular generative model 30.

In this embodiment of this disclosure, the molecular generative model 30 is an ML model configured to generate a molecule. Exemplarily, the molecular generative model 30 is an ML model configured to generate a pharmaceutical molecule. For example, the molecular generative model 30 may output, based on a set property value or a property interval of a target property, a generated molecule that conforms to the target property. In some embodiments, the model training device 10 may train the molecular generative model 30 through ML, to enable the model to possess better performance. The target property may refer to any one or more of molecular properties corresponding to training data (for example, incomplete labeled data), which is not limited in the Embodiments of this disclosure.

The foregoing trained molecular generative model 30 may be deployed in the model using device 20 for use, to provide a molecule generation service. The model using device 20 may be a terminal device such as a mobile phone, a computer, a smart television, a multimedia playback device, a wearable device, or a medical device, or may be a server, which is not limited in the Embodiments of this disclosure.

In some embodiments, as shown in FIG. 1, the molecular generative model 30 may include a first generative network 31, a second generative network 32, and a molecular property prediction network 33.

The molecular property prediction network 33 may be included in the molecular generative model 30, or may not be included in the molecular generative model 30, but serves as a molecular property prediction model alone. The molecular property prediction network 33 may be implemented as the foregoing predictor.

The first generative network 31 and the second generative network 32 are ML networks. In some embodiments, the first generative network 31 is an encoding network, for example, implemented as the foregoing encoder. The second generative network 32 is a decoding network, for example, implemented as the foregoing decoder.

In some embodiments, molecular property prediction data of the molecule is obtained through the molecular property prediction network 33 to fill in a missing labeled property value in the training data. The first generative network 31 and the second generative network 32 are trained based on the filled training data and a sample molecule. In addition, a training process of the molecular generative model is an iteration process. During the iteration, parameters of the first generative network 31, the second generative network 32, and the molecular property prediction network 33 may be continuously adjusted. Therefore, the same sample molecule with a missing labeled property value may be configured for training the first generative network 31 and the second generative network 32 again in combination with the molecular property prediction data obtained by the adjusted molecular property prediction network 33. In some embodiments, an end condition of the foregoing iteration may be that a loss function value of the molecular generative model is less than or equal to a threshold, the loss function value of the molecular generative model is nearly stable, a quantity of iterations of the molecular generative model is greater than or equal to the threshold, and the like, which is not limited in the Embodiments of this disclosure.

In some embodiments, the trained molecular generative model (i.e., the trained molecular generative model) may output a generative molecule having a set property value of the target property.

FIG. 2 is a schematic diagram showing a method for training a molecular generative model according to an embodiment of this disclosure.

As shown in FIG. 2, incomplete labeled data 200 (i.e., training data) includes 6 molecules and labeled property values of the 6 molecules for 3 properties. The labeled property value refers to a value corresponding to a molecular property, such as a numerical value, a grade, and a threshold. In the incomplete labeled data 200, if a part of the labeled property value exists, the part is considered as the labeled data, and if another part of the labeled property value does not exist, the part is considered that the data is missing. Exemplarily, if the incomplete labeled data includes N pieces of molecular property label data, and each piece of molecular property label data includes a molecular labeled property value for M properties, at least one piece of incomplete molecular property label data exists in the N pieces of molecular property label data. The labeled property value of at least one property among the labeled property values of the M properties included in the incomplete molecular property label data is missing, N and M being both positive integers. For example, in the incomplete labeled data 200, a molecule 1 is used as an example. Among the labeled property values of the molecule 1 for the 3 properties, only a value for the property 1 is missing, and values for a property 2 and a property 3 are missing. In some embodiments, such incomplete labeled data may also be referred to as dirty data, and the dirty data is very unfavorable for model training. Moreover, a large number of models do not support training using the dirty data, for example, the foregoing SSVAE model.

Considering that missing data in the incomplete labeled data may affect subsequent model training, in the technical solutions provided in this application, the missing data (such as all or part) in the incomplete labeled data is filled in with the molecular property prediction data, so that the incomplete labeled data 200 may be converted into a relatively complete training sample set 210. It is not difficult to see that in the training sample set 210, values of different molecules for the 3 properties are either the molecular property label data or the molecular property prediction data, and no data is missing.

Therefore, the molecular generative model is trained by using the relatively complete training sample set 210, so as to make up for the shortcomings caused by the dirty data to a great extent, so that the incomplete labeled data is transformed into relatively complete training data. Due to the diversity of the incomplete labeled data, the transformed training sample set has richer data types (such as molecular properties) than another similar training sample set of the complete labeled data. Therefore, diversity of generated results of the molecular generative model may be further improved.

In some embodiments, the molecular generative model trained through the technical solutions of the Embodiments of this disclosure may quickly generate a large number of semantically correct SMILES molecular structures (i.e., molecular data for representing the molecule) based on given constraints (such as specifying several properties required to generate the molecule).

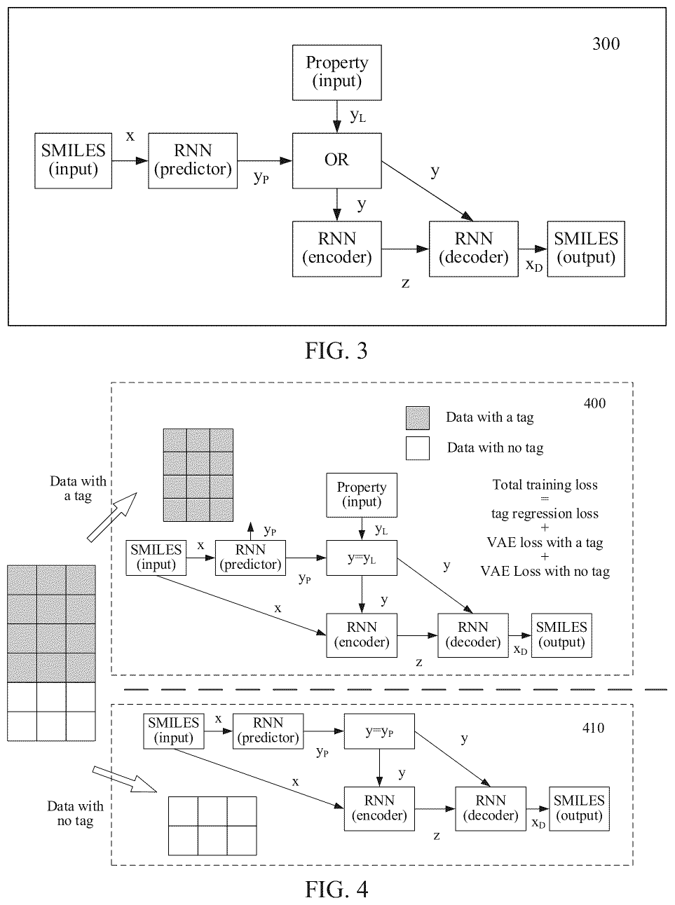

FIG. 3 is a schematic diagram of a method for training an SSVAE model in the related art. 300 of FIG. 3 shows a training architecture for a molecular generative model (namely, the SSVAE model). The SSVAE model is a conditional molecular generative model based on an SSVAE. Main idea of the SSVAE model is as follows. An input molecular structure x from a training dataset (which may also be referred to as training data) is encoded into a latent space representation z by using an encoder sub-model. A predicted property y_{P} of the input molecular structure x is obtained by using a predictor sub-model. If an actual molecular property tag y_{L} exists in the input molecular structure x, y_{P} is discarded, and the model uses the actual molecular property tag y=y_{L}. Otherwise, the predicted property y=y_{P} of the input molecular structure x is used as a molecular property tag of the input molecular structure x. An internal molecular property tag y (i.e., the target property specified above) and the latent space representation z are used as an input to a decoder sub-model to generate an output molecular structure x_{D}.

For the training data (labeled and unlabeled) of different types, the SSVAE model processes data of different types in different manners. For example, the training dataset in each small batch is divided into two different small batches, for example, a small batch with complete labeled data and a small batch without labeled data. Then the SSVAE model is run twice in slightly different modes of operation, depending on whether the small batch is completely labeled or completely unlabeled. For the sake of clarity, reference is made to FIG. 4.

FIG. 4 is a schematic diagram of a method for processing labeled data and unlabeled data by an SSVAE model in the related art. 400 shows a method for training the SSVAE model by using completely labeled data, and 410 shows a method for training the SSVAE model by using completely unlabeled data. All molecules in the completely unlabeled data have no labeled property values, but only predicted property values.

A loss function of a VAE is respectively calculated for the completely unlabeled data and the completely labeled data, and a loss function of a regression task is calculated only for the completely labeled data. Then the three loss functions are added together to calculate a total training loss function for the small batch. Finally, once the SSVAE model is trained, a decoder sub-model in the SSVAE model may be independently run by specifying a property y and a randomly sampled latent spatial input z, to conditionally generate required molecular data.

A main disadvantage of the related art is that the training dataset needs to be completely labeled or completely unlabeled. The SSVAE model splits the problem as shown in FIG. 4, so as to greatly simplify a model data flow, mathematical background, and model behaviors. In practice, the training dataset may include a molecule with an incomplete tag, for example, the completely labeled molecule or the completely unlabeled molecule in FIG. 4. This is especially true if the database for training the molecule is obtained from a publicly available database (such as PubChem) or from a combination of several different databases. FIG. 5 illustrates the case that is more likely to occur.

In some embodiments, there are two cases in which incomplete labeled data appears. One case is that an actual publicly available database is incomplete. As shown in a of FIG. 5, a database may have data with a tag (labeled data) and data without a tag (data missing). Therefore, the database is incomplete and therefore defective (“dirty”). In most databases, a molecular entry with an incomplete property tag (namely, data missing) exists. The other case is that when different databases are merged, a dataset obtained through the merging is incomplete. As shown in b of FIG. 5, a database 1 includes only labeled property values of a molecule 1 to a molecule 3 for a property 1, and a database 2 includes labeled property values of the molecule 3 to a molecule 6 for a property 2 and a property 3. The database 1 and the database 2 are combined to obtain property tags of the molecule 1 to the molecule 6 for the property 1 to the property 3. However, the property tags after the combination are incomplete, among which some labeled property values are missing. For example, the molecule 1 to the molecule 2 lack the labeled property values for the property 2 and the property 3, and the molecule 4 to the molecule 6 lack the labeled property values for the property 1.

In other words, even for an ideal completely labeled molecular property dataset (for example, a molecular property tag obtained by using a calculation method), once the molecular property datasets from different database sources are merged for model training, problems of incomplete labeled data with varying degrees of severity are also encountered. None of the “dirty” (incomplete) datasets in the actual cases can be configured for training the SSVAE model in the related art, thereby severely limiting a usage scenario of the SSVAE model, especially when multi-property conditional molecular generation is required, for example, virtual screening of battery electrolytes or drugs.

However, in the technical solutions provided in the Embodiments of this disclosure, the SSVAE model in the related art is modified to obtain a ConGen model. The model is specifically designed to process the “dirty” (incomplete) training data, so that the ConGen model can be trained by using a large number of training data sources, for example, data sources merged from different public and private sources. Therefore, the ConGen model can complete a conditional molecular generation task that the SSVAE model cannot complete. For example, a completely labeled molecular property label dataset from ZINC (for example, including molecular weight MolWt, hydrophobicity LogP, and drug-likeness QED) and another similar completely labeled molecular property label dataset from Material Project or Electrolyte Genome (for example, including molecular weight MolWt, electron affinity EA, and ionization potential IP) are given to jointly train a molecular generative model (for example, the ConGen model). The molecular generative model may generate a molecule having required values of MolWt, LogP, and EA (which are configured for screening known useful properties of lithium battery electrolytes) (a molecule generated through a model in this embodiment of this disclosure is not necessarily a real molecule. For example, the molecule may be molecular data for describing the molecule. The molecule and the molecular data may be collectively referred to as a molecule in the Embodiments of this disclosure). For these different molecular property label datasets, the SSVAE model needs to process each labeled property separately, namely, MolWt, LogP, and EA. However, the molecular generative model trained by using the technical solutions provided in the Embodiments of this disclosure has no such limitations and allows a user to mix non-ideal actual data from a plurality of sources as needed.

According to the technical solutions provided in the Embodiments of this disclosure, multi-property conditional molecular generation can be learned from a dirty dataset, and the molecular generative model can also be trained by combining a plurality of datasets from different sources. In this way, an incomplete part of the molecular property dataset cannot be ignored, and the missing labeled property value may be filled in the whole training process. Therefore, the learned molecular generative model may satisfy the multi-conditional molecular generation, rather than being limited to only the molecular property condition in the same database, thereby improving diversity of molecular generation.

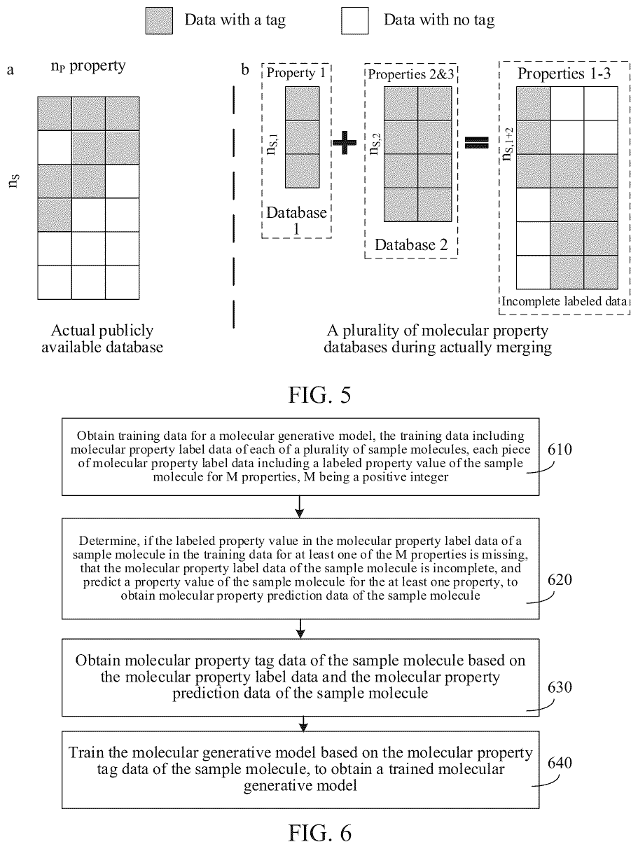

FIG. 6 is a flowchart showing a method for training a molecular generative model according to an embodiment of this disclosure. Each operation of the method may be performed by the model training device described above. In the following method embodiments, for ease of description, that each operation is performed by a “computer device” is used for description. The method may include at least one of the following operations (610-640).

Operation 610: Obtain training data for a molecular generative model, the training data including molecular property label data of each of a plurality of sample molecules, each piece of molecular property label data including a labeled property value of the sample molecule for M properties, M being a positive integer.

The training data refers to data configured for training the molecular generative model. The molecular generative model is an ML model configured to generate a molecule (or molecular data). A specific type of the molecular generative model is not limited in the Embodiments of this disclosure. In some embodiments, the molecular generative model may be the molecular generative model described in the foregoing embodiments. The molecular generative model may also be the foregoing ConGen model. The molecular generative model may also be an adjusted ConGen model (such as a ConGen model with a replaced encoder). The molecular generative model may further be another model for generating the molecule.

In some embodiments, the molecule is in one-to-one correspondence with a molecular representation. The molecular representation includes at least one of the following: a SMILE string, a molecular diagram, a three-dimensional structure of a molecule, and a chemical fingerprint of the molecule. In some embodiments, the foregoing training data may also include labeled property values of the molecular representations of different sample molecules under M properties. In some embodiments, the foregoing training data may also include labeled property values of the SMTLE strings of different sample molecules under M properties.

The sample molecule refers to a molecule serving as a sample for training the molecular generative model. In this application, a quantity of sample molecules delivered to the molecular generative model for training each time is not limited, which may be set and adjusted based on an actual usage requirement. The molecular property label data of the sample molecule is configured for indicating a property of the sample molecule and a labeled property value of the property.

For example, referring to FIG. 2, the training data 200 includes molecular property label data corresponding to each of 6 sample molecules. A 1^{st} piece of molecular property label data includes labeled property values of the molecule 1 for the property 1, the property 2, and the property 3. However, the labeled property values of the molecule 1 for the property 2 and the property 3 are missing. Therefore, if the labeled property value of at least one property among the labeled property values of a molecule for M properties is missing, it may be considered that the molecular property label data of the molecule is incomplete. Among N pieces of molecular property label data, it is considered that the entire data is incomplete labeled data as long as one piece of molecular property label data is incomplete. In other words, the training data 200 may also be referred to as the incomplete labeled data. Simply put, it may also be considered that as long as one labeled property value in a molecular property label dataset with a scale of N*M is missing, it is considered that the molecular property label dataset is the incomplete labeled data. Correspondingly, if any labeled property value is present in the molecular property label dataset with the scale of N*M, it is considered that the dataset is the complete labeled data, N being a quantity of molecules corresponding to the training data, namely, a total quantity of sample molecules.

In some embodiments, the properties mentioned in the Embodiments of this disclosure all refer to the molecular properties, namely, properties possessed by the molecule. In some embodiments, the molecular property includes at least one of the following: molecular weight MolWt, hydrophobicity LogP, drug-likeness QED, electron affinity EA, and ionization potential IP. In some embodiments, a quantity of types of the molecular properties in the complete labeled data is less than a quantity of types of the molecular properties in the incomplete labeled data. As shown in FIG. 5, only one molecular property is present in a database 1, and only a molecular property 2 and a molecular property 3 are present in a database 2. However, the incomplete labeled data 200 (namely, the training data) in FIG. 2 has 3 molecular properties.

In some embodiments, the training data is constructed based on the incomplete labeled data in the Embodiments of this disclosure. For example, all or part of the incomplete labeled data is directly determined as the training data. For example, the sample molecule included in the training data may be all or part of N molecules corresponding to the incomplete labeled data. In some embodiments, the incomplete labeled data may be generated by merging the complete labeled data. Therefore, the quantity of types of the molecular properties in the incomplete labeled data is greater than the quantity of types of the molecular properties in the complete labeled data. In other words, the incomplete labeled data has a larger scale. In some other embodiments, the incomplete labeled data is a large database. However, due to a wide variety of molecular properties and a large quantity of molecules, the data in the database is incomplete and relatively dirty. Certainly, although the incomplete labeled data is not complete, compared with the complete labeled data, the incomplete labeled data may have a larger data scale, more types of the molecular properties, and a larger quantity of molecules.

The foregoing labeled property value is configured for labeling a property of a molecule. The labeled property value may refer to a real property value, or may refer to a property value labeled by an expert, and may further refer to a property value determined by a mature model, which is not limited in the Embodiments of this disclosure. Exemplarily, the labeled property value among the molecular property label data may be data related to the property value of the property of the sample molecule recorded in an authoritative database. The data may be measured by experts and scholars, or may be predicted through a high-precision molecular property prediction model. A manner of obtaining the molecular property label data is not limited in the Embodiments of this disclosure. For example, a large molecular property label dataset ZINC includes labeled property values of a molecule for three molecular properties including the molecular weight MolWt, the hydrophobicity LogP, and the drug-likeness QED. Another similar large-scale molecular property label dataset, namely, Materials Project or Electrolyte Genome, includes labeled property values of a molecule for three molecular properties including the molecular weight MolWt, the electron affinity EA, and the ionization potential IP. In some embodiments, the molecular property label data regarding all of the molecular weight MolWt, the hydrophobicity LogP, the drug-likeness QED, the electron affinity EA, and the ionization potential IP may be constructed based on the molecular property label data in the ZINC and the Materials Project or Electrolyte Genome. However, the obtained molecular property label data is incomplete. To be specific, some labeled property values are missing. A process of obtaining the incomplete labeled data (i.e., the training data) is to be described in detail below. Details are not described herein again.

Operation 620: Determine, if the labeled property value in the molecular property label data of a sample molecule in the training data for at least one of the M properties is missing, that the molecular property label data of the sample molecule is incomplete, and predict a property value of the sample molecule for the at least one property, to obtain molecular property prediction data of the sample molecule.

In this embodiment of this disclosure, label completeness of the training data is determined first. In a case that a label of the training data is complete, the molecular generative model may be directly trained based on the training data. In a case that the label of the training data is incomplete, the molecular generative model may be trained based on the molecular property label data and the molecular property prediction data.

The molecular property prediction data refers to data predicted for the property of the molecule. The molecular property prediction data may include at least one predicted property value. The predicted property value is a predicted value, for example, a property value predicted for the property, and the labeled property value is a real value of the property value. A scale of the molecular property prediction data is not limited in the Embodiments of this disclosure. In some embodiments, the molecular property prediction data of a molecule may include predicted property values of the molecule for the M properties. In some embodiments, the molecular property prediction data of a molecule may include the predicted property value of the molecule for the foregoing at least one property. FIG. 2 is used as an example. The molecular property prediction data of the molecule 1 may include the predicted property values only for the property 2 and the property 3. Through prediction of only missing data, training costs can be reduced to a great extent. A method for obtaining the molecular property prediction data is to be described in detail below. Details are not described herein again.

In some embodiments, the foregoing sample molecule may refer to any sample molecule in the training data, and the at least one property may refer to any one or more of the M properties.

Operation 630: Obtain molecular property tag data of the sample molecule based on the molecular property label data and the molecular property prediction data of the sample molecule.

The molecular property tag data: It is used as tag data to guide training of the molecular generative model, which may include property values (the labeled property value+the predicted property value) of the sample molecule for the M properties. For a sample molecule, missing data in the molecular property label data may be filled in with the molecular property prediction data based on the molecular property label data of the sample molecule, to obtain molecular property tag data of the molecule. A scale of the molecular property tag data of any sample molecule is M.

For example, if the molecular property label data corresponding to the sample molecule is incomplete, it indicates that the labeled property value corresponding to the sample molecule is missing. A labeled property value that is not missing is retained to fill in the missing labeled property value with the predicted property value, so that the molecular property tag data corresponding to the sample molecule may be obtained. If the molecular property label data corresponding to the sample molecule is complete, the molecular property label data corresponding to the sample molecule may be directly used as the molecular property tag data corresponding to the sample molecule.

Operation 640: Train the molecular generative model based on the molecular property tag data of the sample molecule, to obtain a trained molecular generative model.

In some embodiments, the trained molecular generative model may be a molecular generative model that has been trained, which may be configured to generate molecular data having at least one of the M properties. For example, the molecular generative model that has been trained may be configured to generate molecular data having a target property. The target property may refer to any one or more of the M properties. The target property may be set and adjusted based on an actual usage requirement, which is not limited in the Embodiments of this disclosure. The molecular data refers to data configured for describing a generated molecule, for example, the foregoing SMILES string. The generated molecule is an output of the molecular generative model. The molecule corresponding to the molecular data has the target property. The molecular generative model that has been trained may refer to the molecular generative model whose model parameter has been adjusted, for example, a ConGen model whose model parameter has been adjusted.

In some embodiments, the sample molecule with complete molecular property label data in the training data is denoted as a first-type sample molecule, and the sample molecule with incomplete molecular property label data in the training data is denoted as a second-type sample molecule. In this way, the molecular generative model may be trained by using only the second-type sample molecule and the molecular property tag data (including the predicted property value) corresponding to the second-type sample molecule. For example, iterative training is performed on the molecular generative model by using one second-type sample molecule as an input to one iteration. For another example, the iterative training is performed on the molecular generative model by using a plurality of second-type sample molecules as an input to one iteration, which is not limited in the Embodiments of this disclosure.

Exemplarily, the sample molecule (namely, the second-type sample molecule) and the molecular property tag data corresponding to the sample molecule may be used as training samples to form a training sample set, and the iterative training is performed on the molecular generative model by using the training sample set, to obtain the trained molecular generative model.

For example, the molecular generative model is trained by using the molecular representation corresponding to the sample molecule and the tagged property value of the sample molecule in the molecular property tag data, to obtain the trained molecular generative model. In some embodiments, the generated molecule corresponding to the sample molecule is obtained based on the molecular property tag data through the molecular generative model, and then a loss function value is determined based on a difference between the sample molecule and the generated molecule. Finally, the parameter of the molecular generative model is adjusted by using the loss function value, to obtain the trained molecular generative model. A specific model architecture of the molecular generative model and a specific model training process are not limited in the Embodiments of this disclosure.

In some embodiments, the training data may further be filled with the molecular property tag data of the second-type sample molecule, to obtain filled training data, and then the molecular generative model is trained by using the filled training data, to obtain the trained molecular generative model.

In some embodiments, the training data may be filled in units of the molecular property label data, or the training data may be filled in units of the property value.

In an example, the training data may be filled with the molecular property tag data corresponding to all of the second-type sample molecules corresponding to the training data, to obtain complete training data. The molecular generative model is trained through the complete training data. For example, a plurality of pieces of mini-batch data is constructed based on the complete training data. The iterative training is performed on the molecular generative model through the plurality of pieces of mini-batch data, to obtain the trained molecular generative model. For another example, the complete training data is an input to one iteration. After a plurality of pieces of complete training data is constructed, the iterative training is performed on the molecular generative model through the plurality of pieces of complete training data.

In an example, the training data may be filled with the molecular property tag data corresponding to a part (including one) of the second-type sample molecules corresponding to the training data, to obtain the filled training data. The molecular generative model is trained through the filled training data. For example, after the filled training data is obtained, the sample molecule in the filled training data that still has incomplete molecular property label data is eliminated, to obtain final training data. Then the plurality of pieces of mini-batch data is constructed based on the final training data, and the iterative training is performed on the molecular generative model through the plurality of pieces of mini-batch data. Alternatively, the final training data is used as the input to one iteration. After the plurality of pieces of final training data is constructed, the iterative training is performed on the molecular generative model through the plurality of pieces of final training data. Construction costs of the training data may be effectively reduced by filling only part of the training data, thereby reducing the training costs of the molecular generative model.

In some embodiments, an end condition of the foregoing iteration may be that a loss function value of the molecular generative model is less than or equal to a threshold, the loss function value of the molecular generative model is nearly stable, a quantity of iterations of the molecular generative model is greater than or equal to the threshold, and the like, which is not limited in the Embodiments of this disclosure.

Further, except for the molecular generative model, a training sample set of another model may also be constructed by using the technical solution provided in this embodiment of this disclosure. In other words, the training sample including both the labeled data and the predicted data is constructed by filling a data missing position in the incomplete labeled data with the predicted data, so as to improve diversity of predicted results and mix the plurality of pieces of complete labeled data to train the models together without the need to train the models one by one, thereby improving training efficiency of the model.

According to the technical solutions provided in the Embodiments of this disclosure, the molecular generative model is trained by using the training data with a missing labeled property value, so that newly generated molecular data has more abundant molecular properties that are not limited to a few molecular properties specified in complete labeled data, thereby improving diversity of the generated molecular data. In addition, the training data may include a plurality of pieces of complete labeled data. Therefore, types of the molecular properties of the sample molecule can be enriched through the training data, thereby further improving diversity of output results of the molecular generative model.

In addition, since the labeled property values for the M properties in the training data are missing, a data gap in incomplete labeled data is filled in by obtaining the molecular property prediction data corresponding to the sample molecule, so as to improve completeness of the training sample, achieve a smoother subsequent training process, and facilitate improvement in training accuracy of the molecular generative model.

In addition, different from a training manner in which the molecular generative model is trained by using only the complete labeled data in the related art, in the technical solutions provided in the Embodiments of this disclosure, the training data with a missing labeled property value may also be configured for training the molecular generative model. Therefore, the method for training a molecular generative model is also enriched.

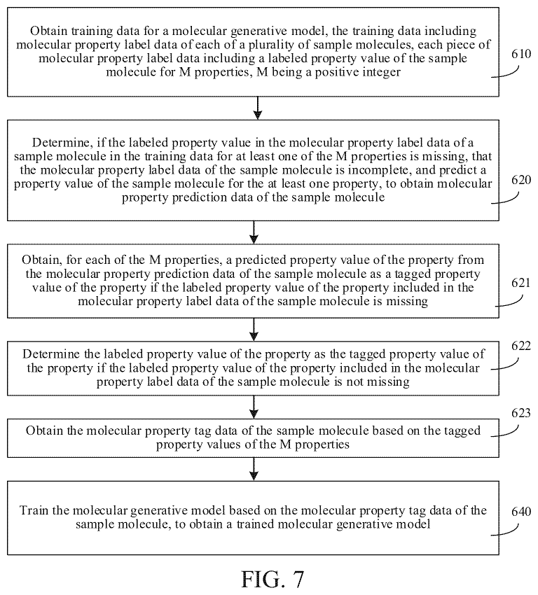

FIG. 7 is a flowchart showing a method for training a molecular generative model according to another embodiment of this disclosure. Each operation of the method may be performed by the model training device described above. In the following method embodiments, for ease of description, that each operation is performed by a “computer device” is used for description. The method may include at least one of the following operations (610-640).

Operation 610: Obtain training data for a molecular generative model, the training data including molecular property label data of each of a plurality of sample molecules, each piece of molecular property label data including a labeled property value of the sample molecule for M properties, M being a positive integer.

In some embodiments, operation 610 includes at least one of operation 611 to operation 612 (not shown in the figure).

Operation 611: Obtain at least two sets of completely labeled data, each set of completely labeled data including a labeled property value of at least one molecule for the at least one property, properties included in different sets of completely labeled data being different, and molecules included in the different sets of completely labeled data being different.

The completely labeled data herein corresponds to the complete labeled data described above. In other words, the labeled property value of the molecule in the completely labeled data for any one of the properties is not missing. As shown in b of FIG. 5, the data in the database 1 and the data in the database 2 both belong to the completely labeled data. The database 1 includes only labeled property values of the molecule 1 to the molecule 3 for the property 1. The database 2 includes labeled property values of the molecule 3 to the molecule 6 for the property 2 and the property 3. A difference exists in the properties included in the database 1 and the database 2. The molecules included are also different.

In some embodiments, part of the completely labeled data may also be selected from one piece of complete labeled data, and part of the completely labeled data is also selected from another piece of complete labeled data. This is due to the consideration of a molecular property of interest. If not all molecular properties in a large database are to be learned, a required molecular property or the molecular property of interest is selected from the database, and partial data is selected as the completely labeled data. In other words, two complete databases are not necessarily to be merged, or partial data in the two complete databases may be merged. In this way, the training of the molecular generative model may be more targeted, and training costs of the molecular generative model may further be reduced.

In some embodiments, for at least two sets of completely labeled data that need to be integrated, different sets of completely labeled data include different properties, and the different sets of completely labeled data include different molecules, so as to further improve diversity of the integrated incomplete labeled data and facilitate subsequent generation of a conditional molecule. Since the molecular generation requires relatively great uncertainty, completely fixed training is likely to cause a failure to find a suitable molecule to be generated, or the generated molecules are all the same. As a result, the existence of the molecular generative model is meaningless. However, according to the technical solutions provided in the Embodiments of this disclosure, at least two sets of completely labeled data are integrated to obtain the incomplete labeled data. The randomness of the property types of the incomplete labeled data is relatively high, and the randomness of the molecules is also relatively high, so as to facilitate training of such a random molecular generative model, thereby improving generative capacity of the molecular generative model.

Operation 612: Integrate the at least two sets of completely labeled data at a granularity of each molecule, to obtain the training data.

The molecules are arranged in a first order. The molecules are integrated first (for example, de-duplicated and arranged), and then the labeled property values corresponding to the molecules are integrated (for example, all labeled property values corresponding to the molecules are summarized). For example, as shown in b of FIG. 5, at least two sets of completely labeled data are integrated at a granularity of each molecule. For example, a molecule 3 in a third row of the database 1 and a molecule 3 in a first row of the database 2 are integrated together, and other molecules are integrated correspondingly. The integration includes the following. For each molecule, the labeled property value is retained at a property position where the molecule has a corresponding labeled property value, and a property position for the molecule with no labeled property value is set to null, to obtain the training data (namely, the incomplete labeled data).

In the technical solutions provided in the Embodiments of this disclosure, the completely labeled data is integrated at a granularity of a molecule, so as to ensure accuracy of the integrated training data and avoid data confusion as a result of integration of a plurality of sets of completely labeled data.

In some embodiments, after operation 610, operation 615 is further included (not shown in the figure).

Operation 615: Generate a mask matrix corresponding to the training data, the mask matrix including N×M elements, each element being configured for indicating whether a labeled property value of a property of a sample molecule is missing, and N being a positive integer.

The mask matrix corresponds to the training data, and may be configured for instructing the molecular generative model to extract the labeled property value in the training data. For example, values corresponding to an i^{th} row and a j^{th} column in the mask matrix may be configured for indicating whether an i^{th} molecule in the training data has a labeled property value for a j^{th} property. If the value is 1, the i^{th} molecule has the labeled property value for the j^{th} property. If the value is 0, the i^{th} molecule has no labeled property value for the j^{th} property.

In some embodiments, the mask matrix is generated based on the training data. In some embodiments, a size of the mask matrix is related to a quantity of molecules and a quantity of properties of the training data. In some embodiments, if the training data includes N molecules and M properties, the size of the mask matrix is N×M.

In some embodiments, the mask matrix is generated in the following two manners.

The first manner is that in the mask matrix, numerical values of all positions corresponding to the foregoing at least two sets of completely labeled data in the training data are set to a first numerical value, and numerical values of all positions not corresponding to the at least two sets of completely labeled data in the training data are set to a second numerical value. In other words, the first numerical value is configured for characterizing that the property value corresponding to the position is the labeled property value, and the second numerical value is configured for characterizing that the property value corresponding to the position is missing and needs to be filled with a predicted property value.

The second manner is it is determined element by element in the mask matrix whether the position has a corresponding labeled property value in the training data. If the position has the labeled property value, the value of the position is set to the first numerical value, and if the position has no labeled property value, namely, data of the position is missing, the value of the position is set to the second numerical value.

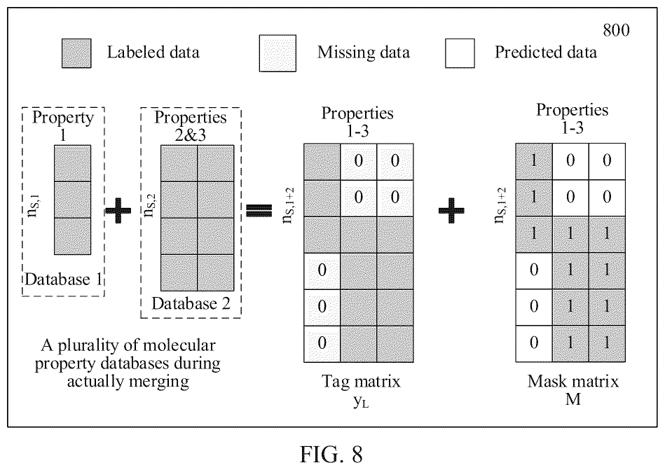

FIG. 8 is a schematic diagram showing a mask matrix according to an embodiment of this disclosure. 800 shows a determined architecture of the mask matrix. The architecture of the mask matrix is the same as an architecture of a tag matrix (i.e., training data). A value of a position having a labeled property value in the tag matrix is set to 1, and a value of a position having no labeled property value in the tag matrix is set to 0, so as to obtain the mask matrix corresponding to the training data.

In some embodiments, a model training device 10 obtains the training data through a training data loader, and the training data loader may be modified, so that the training data loader has the ability to merge databases and process dirty data.

Exemplarily, since the training data loader needs to operate by using an actual training dataset, the training data loader is needed to have the ability to merge the databases (even if a merged molecular database includes different molecular properties) and process a dirty dataset that does not include valid molecular property label data. In some embodiments, the two requirements may be met in the following manners. 1. A molecular SMILES string is normalized by using a Python cheminformatics library such as RDKit (to ensure that the same molecular structure shares the same SMILES string). 2. A new tag matrix y_{L} is constructed. All of the molecular SMILES strings involved in different databases are used as a row, and all properties involved in different databases are used as a column. Then a corresponding labeled property value is copied from a source database as a valid property entry in a matrix y_{L}. 3. A mask matrix M of the same size as y_{L} is constructed, where 1 is configured for characterizing the valid property entry, and 0 is configured for characterizing an invalid property entry.

Based on the mask matrix corresponding to the training data, the molecular generative model can determine which property data needs to be filled in and which property data is valid in the training data, and can distinguish between the labeled property value and the predicted property value in the molecular property tag data. Therefore, the molecular generative model has the ability to process the dirty dataset, thereby reducing training costs of the molecular generative model.

Operation 620: Determine, if the labeled property value in the molecular property label data of a sample molecule in the training data for at least one of the M properties is missing, that the molecular property label data of the sample molecule is incomplete, and predict a property value of the sample molecule for the at least one property, to obtain molecular property prediction data of the sample molecule.

Operation 610, operation 602, and operation 640 are the same as those described in the foregoing embodiments. For content not described in this embodiment of this disclosure, reference may be made to the foregoing embodiments. Details are not described herein again.

Operation 621: Obtain, for each of the M properties, a predicted property value of the property from the molecular property prediction data of the sample molecule as a tagged property value of the property if the labeled property value of the property included in the molecular property label data of the sample molecule is missing.

A data gap in incomplete labeled data is filled with the predicted property value, so as to improve completeness of the training sample, achieve a smoother subsequent training process, and facilitate improvement in training accuracy of the molecular generative model.

Operation 622: Determine the labeled property value of the property as the tagged property value of the property if the labeled property value of the property included in the molecular property label data of the sample molecule is not missing.

Operation 623: Obtain the molecular property tag data of the sample molecule based on tagged property values of M properties.

Operation 640: Train the molecular generative model based on the molecular property tag data of the sample molecule, to obtain a trained molecular generative model.

The trained molecular generative model may be configured to generate molecular data with a target property. The target property may refer to at least one of the M properties. The molecular data may refer to a molecular representation for describing a molecular structure of a generated molecule, for example, an SMILE string, a molecular diagram, a three-dimensional structure of a molecule, and a chemical fingerprint of the molecule.

In some embodiments, the molecular generative model can process the dirty data (namely, the incomplete labeled data) through the mask matrix and the molecular property tag data, and then the molecular generative model may be directly trained by using the molecular property tag data of the sample molecule without the need to divide data into completely labeled data and completely unlabeled data. The completely labeled data and the completely unlabeled data are separately processed to train the molecular generative model.

Exemplarily, as shown in FIG. 3, in the related art, when an SSVAE model processes the completely labeled data, the tag matrix y_{L} is used as an input to an encoder and a decoder. However, when the SSVAE model processes the completely unlabeled data, a predictor outputs a predicted property value y_{P} to be used as the input to the encoder and decoder, which may be expressed by using the following equation:

$$y ( i , j ) = \{ \begin{matrix}{y}_{L} ( i , j ) & if M ( i , j ) == 1 \\ {y}_{P} ( i , j ) & if M ( i , j ) == 0\end{matrix} , \\ where i represents a molecule , and j represents a property .$$

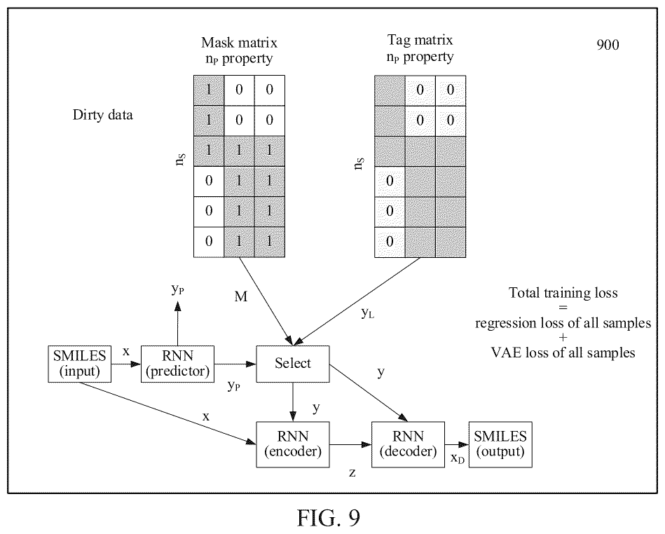

In the technical solutions provided in the Embodiments of this disclosure, the molecular generative model and the SSVAE model in this embodiment of this disclosure have the same input-output relationship through the mask matrix. To be specific, the molecular generative model can automatically identify the labeled property value and the predicted property value based on the mask matrix, and can automatically identify the generated molecule corresponding to each of the labeled property value and the predicted property value. In some embodiments, the molecular generative model in the Embodiments of this disclosure is a ConGen model. In some embodiments, an implementation principle of the ConGen model is shown in 900 of FIG. 9. The dirty data (incomplete labeled data) is processed by using the mask matrix, and after obtaining the molecular property tag data of the sample molecule, the ConGen model uses the mask matrix to select a tagged property value y (for example, 1 corresponds to the labeled property value, and 0 corresponds to the predicted property value) for a molecular representation x corresponding to the sample molecule from the molecular property tag data, to obtain a latent space representation z corresponding to the sample molecule. Then the ConGen model uses the latent space representation z and the tagged property value y to obtain a generated molecule X_{D} (such as the SMILES string) with the tagged property value y corresponding to the sample molecule.

In the technical solutions provided in the Embodiments of this disclosure, the mask matrix is introduced to enable the molecular generative model to process the labeled data and the unlabeled data simultaneously. To be specific, the molecular generative model may process the incomplete labeled data, without needing to separate the two to process the completely labeled data and the completely unlabeled data separately. Therefore, in the technical solutions provided in the Embodiments of this disclosure, the processing of the incomplete labeled data is simpler, thereby improving processing efficiency of the molecular generative model, and reducing the training costs of the molecular generative model.

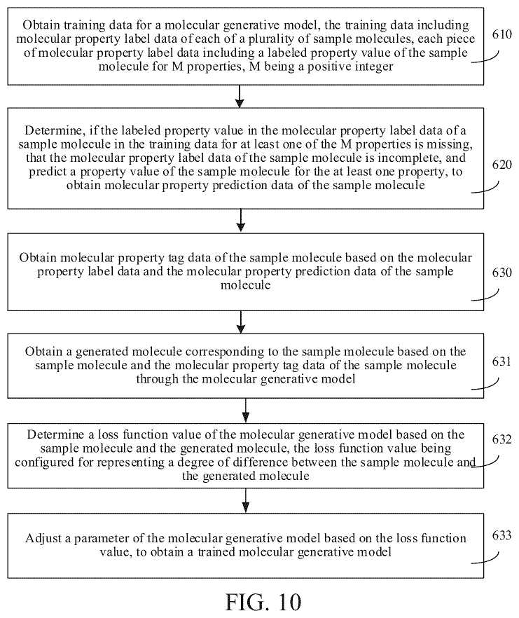

FIG. 10 is a flowchart showing a method for training a molecular generative model according to another embodiment of this disclosure. Each operation of the method may be performed by the model training device described above. In the following method embodiments, for ease of description, that each operation is performed by a “computer device” is used for description. The method may include at least one of the following operations (610-633).

Operation 610: Obtain training data for a molecular generative model, the training data including molecular property label data of each of a plurality of sample molecules, each piece of molecular property label data including a labeled property value of the sample molecule for M properties, M being a positive integer.

Operation 620: Determine, if the labeled property value in the molecular property label data of a sample molecule in the training data for at least one of the M properties is missing, that the molecular property label data of the sample molecule is incomplete, and predict a property value of the sample molecule for the at least one property, to obtain molecular property prediction data of the sample molecule.

Operation 630: Obtain molecular property tag data of the sample molecule based on the molecular property label data and the molecular property prediction data of the sample molecule.

Operation 610, operation 620, and operation 630 are the same as those described in the foregoing embodiments. For content not described in this embodiment of this disclosure, reference may be made to the foregoing embodiments. Details are not described herein again.

Operation 631: Obtain a generated molecule corresponding to the sample molecule based on the sample molecule and the molecular property tag data of the sample molecule through the molecular generative model.

The generated molecule refers to an output obtained by the molecular generative model by processing the sample molecule, which may be a simulation of the sample molecule under a conditional property. In some embodiments, if the input of the molecular generative model is a molecular representation of the sample molecule, the output of the molecular generative model is also a molecular representation of the generated molecule. Exemplarily, if the input is the SMILE string of the sample molecule, the output is also the SMILE string of the generated molecule.

Operation 632: Determine a loss function value of the molecular generative model based on the sample molecule and the generated molecule, the loss function value being configured for characterizing a degree of difference between the sample molecule and the generated molecule.

Before the loss function value of the molecular generative model provided in the Embodiments of this disclosure is introduced, a loss function value of an SSVAE model in the related art is introduced.

During execution of the SSVAE model, there is no interaction among the sample molecules in a small batch (for example, if a sample molecule A and a sample molecule B are simultaneously processed, model outputs X_{D} of the two sample molecules do not affect each other), so as to ensure that any internal model variable (y_{L}, y_{P}, z, X_{D}, or the like) of the sample molecule x is completely determined by the sample molecule x. Therefore, the implementation of the loss function value of the molecular generative model in this embodiment of this disclosure becomes relatively simple. In other words, there is also no interaction among the sample molecules in the small batch during the execution of the molecular generative model.

During initial implementation of the loss function value of the SSVAE model, the loss function value of the sample molecule needs to be split into three parts. The three parts are as follows:
a. A VAE loss function value of completely labeled data in the small batch:

$$( x , y ) = - {\sum }_{i = 1}^{{n}_{L}} {\sum }_{j = 1}^{{n}_{x}} ( {x}_{i , j} ln {x}_{D , i , j} + ( 1 - {x}_{i , j} ) ln ( 1 - {x}_{D , i , j} ) ) + {\sum }_{i = 1}^{{n}_{L}} \frac{1}{2} ( {n}_{y} ln 2 π + ln ( det ( C ) ) + {\sum }_{j = 1}^{{n}_{y}} ( {y}_{L , i , j} - {E}_{j} ) {\sum }_{k = 1}^{{n}_{y}} ( {y}_{L , i , k} - {E}_{k} ) {C}_{k , j}^{- 1} ) - {\sum }_{i = 1}^{{n}_{L}} {\sum }_{j = 1}^{{n}_{z}} \frac{1}{2} ( 1 + ln {σ ( {z}_{i , j} )}^{2} - {μ ( {z}_{i , j} )}^{2} - {σ ( {z}_{i , j} )}^{2} )$$

b. A VAE loss function value of completely unlabeled data in the small batch:

$$( x ) = - {\sum }_{i = 1}^{{n}_{U}} {\sum }_{j = 1}^{{n}_{x}} ( {x}_{i , j} ln {x}_{D , i , j} + ( 1 - {x}_{i , j} ) ln ( 1 - {x}_{D , i , j} ) ) + {\sum }_{i = 1}^{{n}_{U}} \frac{1}{2} ( {\sum }_{j = 1}^{{n}_{y}} {C}_{j , j}^{- 1} {σ ( {y}_{P , i , j} )}^{2} + {\sum }_{j = 1}^{{n}_{y}} ( {y}_{P , i , j} - {E}_{j} ) {\sum }_{k = 1}^{{n}_{y}} ( {y}_{P , i , k} - {E}_{k} ) {C}_{k , j}^{- 1} - {n}_{y} + ln ( det ( C ) ) - {\sum }_{j = 1}^{{n}_{y}} ln {σ ( {y}_{P , i , j} )}^{2} ) - {\sum }_{i = 1}^{{n}_{U}} {\sum }_{j = 1}^{{n}_{z}} \frac{1}{2} ( 1 + ln {σ ( {z}_{i , j} )}^{2} - {μ ( {z}_{i , j} )}^{2} - {σ ( {z}_{i , j} )}^{2} )$$

c. A regression loss function value of the completely labeled data in the small batch:

$$( x , y ) = β {\sum }_{i = 1}^{{n}_{L}} {\sum }_{j = 1}^{{n}_{y}} {( {y}_{L , i , j} - μ ( {y}_{P , i , j} ) )}^{2}$$

where C=Cov(y_{L}) and E=E(y_{L}) are a covariance matrix and a mean value of tags obtained through statistics in the training data, μ is a mean value function, σ is a standard deviation function, β is a hyper-parameter for balancing a generated task and supervised learning, n_{L}, n_{U}, nₓ, n_{y}, and n_{z} are respectively the completely labeled data, the completely unlabeled data, and dimensions of x, y, and z (i.e., a total quantity) in a batch of data, x is a sample molecule, y is a labeled property value, z is a latent space representation, x_{i,j} is a j^{th} sample molecule in an i^{th} completely labeled data, x_{D,i,j} is a generated molecule corresponding to x_{i,j}, y_{L,i,j} is a labeled property value corresponding to x_{i,j} in the completely labeled data, z_{i,j} is a latent space representation corresponding to x_{i,j}, and y_{P,i,j} is a predicted property value corresponding to x_{i,j} in the completely unlabeled data. Finally, the loss function of the batch of data is Cost_{SSVAE}= [symbol] + [symbol] + [symbol] .

It may be learned from the loss function above that terms between L and U actually overlap to a great extent. Therefore, in this embodiment of this disclosure, L and U may be combined and simplified based on the incomplete labeled data by using the mask matrix M, to calculate or obtain a loss function value G of the molecular generative model in this embodiment of this disclosure. Exemplarily, when the molecular property label data is completely labeled, entries of M are all 1, and G needs to be converted into L except for some constant terms. When all of the molecular property label data is completely unlabeled, the entries of M are all 0, and G needs to be converted into U except for some constant terms. Similarly, the loss function R corresponding to this embodiment of this disclosure also needs to calculate a sum of entries labeled in the small batch. By ensuring such a behavior, when the completely labeled data or the completely unlabeled data is provided, a differential of the loss function value G and parameter optimization of the molecular generative model are to be exactly the same as results obtained through respectively processing the completely labeled data/completely unlabeled data.

In some embodiments, the loss function value in this embodiment of this disclosure includes a regression loss and a variational encoding loss, the variational encoding loss including a first loss, a second loss, a third loss, and a fourth loss. The regression loss corresponds to the foregoing R, and the variational encoding loss corresponds to the foregoing L+U.

In some embodiments, operation 632 includes at least one of operation 632-1 to operation 632-6 (not shown in the figure).

Operation 632-1: Determine the regression loss based on the labeled property value of the labeled property in the molecular property label data and the predicted property value of the labeled property in the molecular property prediction data, the regression loss being configured for characterizing prediction accuracy of the molecular property prediction data, and the labeled property being a property corresponding to the labeled property value that is not missing in the molecular property label data.

Operation 632-2: Determine the first loss based on the generated molecule and the sample molecule, the first loss being configured for characterizing a degree of direct difference between the generated molecule and the sample molecule. For example, the first loss is determined based on the difference between the molecular representation corresponding to the generated molecule and the molecular representation corresponding to the sample molecule.

Operation 632-3: Determine the second loss and the third loss based on the molecular property tag data, a covariance matrix of the molecular property tag data, a mean value of the molecular property tag data, and a mean value of the molecular property prediction data, the second loss being configured for characterizing a degree of difference between the molecular property tag data and a tagged property value of the labeled property in the molecular property tag data, and the third loss being configured for characterizing a degree of difference between the molecular property tag data a tagged property value of another property among the M properties included in the molecular property tag data other than the labeled property.

Operation 632-4: Determine the fourth loss based on a latent space representation and position parameters and a mean value of a probability distribution to which the latent space representation conforms, the fourth loss being configured for characterizing a degree of dispersion of the latent space representation relative to the probability distribution, the latent space representation being a hidden layer feature of the sample molecule obtained by an intermediate layer of the molecular generative model.

Operation 632-5: Determine the variational encoding loss based on the first loss, the second loss, the third loss, and the fourth loss, the variational encoding loss being configured for characterizing a degree of difference between the sample molecule and the generated molecule generated based on the molecular property tag data.

Operation 632-6: Determine the loss function value based on the regression loss and the variational encoding loss.

A new loss function is defined herein for batch data during training of the molecular generative model (for example, the ConGen model), especially for dirty data. The variational encoding loss and the regression loss may be respectively expressed as follows:
a. A VAE loss function for the dirty data in the small batch:

$$( x , y ) = - {\sum }_{i = 1}^{{n}_{S}} {\sum }_{j = 1}^{{n}_{x}} ( {x}_{i , j} ln {x}_{D , i , j} + ( 1 - {x}_{i , j} ) ln ( 1 - {x}_{D , i , j} ) ) + {\sum }_{i = 1}^{{n}_{S}} \frac{1}{2} ( {n}_{y} ln 2 π + {\sum }_{j = 1}^{{n}_{y}} {M}_{i , j} ( {y}_{i , j} - {E}_{j} ) {\sum }_{k = 1}^{{n}_{y}} {M}_{i , k} ( {y}_{i , k} - {E}_{k} ) {C}_{k , j}^{- 1} ) + {\sum }_{i = 1}^{{n}_{S}} \frac{1}{2} ( {\sum }_{j = 1}^{{n}_{y}} {C}_{j , j}^{- 1} ( 1 - {M}_{i , j} ) {σ ( {y}_{i , j} )}^{2} + {\sum }_{j = 1}^{{n}_{y}} ( 1 - {M}_{i , j} ) ( {y}_{i , j} - {E}_{j} ) {\sum }_{k = 1}^{{n}_{y}} ( 1 - {M}_{i , k} ) ( {y}_{i , k} - {E}_{k} ) {C}_{k , j}^{- 1} - {n}_{y} - {\sum }_{j = 1}^{{n}_{y}} ( 1 - {M}_{i , j} ) ln {σ ( {y}_{i , j} )}^{2} ) - {\sum }_{i = 1}^{{n}_{S}} {\sum }_{j = 1}^{{n}_{z}} \frac{1}{2} ( 1 + ln {σ ( {z}_{i , j} )}^{2} - {μ ( {z}_{i , j} )}^{2} - {σ ( {z}_{i , j} )}^{2} )$$

b. A regression loss function for the dirty data in the small batch:

$$( x , y ) = β {\sum }_{i = 1}^{{n}_{S}} {\sum }_{j = 1}^{{n}_{y}} {{M}_{i , j} ( {y}_{i , j} - μ ( {y}_{P , i , j} ) )}^{2}$$

where n_{S} is a quantity of sample molecules inputted to a model in a small batch, M is the mask matrix, and M_{i,j} is a corresponding numerical value x_{i,j} among M. Compared with the related art, ln(det(C)) is deleted from the VAE loss function, which is mainly a consideration of stability of the numerical value. The final loss function value is Cost_{ConGen}= [symbol] + [symbol] , where [symbol] is the variational encoding loss, [symbol] is the regression loss, and [symbol] includes the first loss, the second loss, the third loss, and the fourth loss that respectively correspond to four parts in the variational encoding loss.

In some embodiments, in a case that the molecular property prediction data of the sample molecule continuously changes with the training of the molecular generative model, the covariance matrix and the mean value of the molecular property tag data are also continuously updated.

In some embodiments, a problem of non-PSD covariance matrix caused by the dirty data may be avoided by using a covariance matrix interpolation method. An example in which the molecular generative model in this application is the ConGen model is used. Operation 632 may further include the following content.

The covariance matrix and the mean value of the molecular property tag data are used as parts of the loss function values of the SSVAE model and the ConGen model, and C=Cov(y_{L}) and E=E(y_{L}) need to be calculated first, which are respectively a tag covariance matrix and a tag mean value constructed based on the whole training set. For the SSVAE model, unlabeled molecular property label data is completely discarded directly from the training set, and C and E are directly calculated based on the completely labeled molecular property label data. The calculation may be performed once during model construction, and the values are set during the whole model training.

However, such a strategy is not applicable to the ConGen model because the training data is dirty. For a tag matrix Y_{L} of the incomplete labeled data, it is meaningful to calculate the tag mean value E only based on an available property entry and ignore missing data in the matrix y_{L}. Similarly, it is more meaningful to ignore the missing data while calculating the term C of the covariance matrix based on the available property entries in the matrix y_{L}. In other words, for the calculation of E and C, there are the following cases:

$${E}_{j} = {E ( {y}_{L} )}_{j} = \frac{{\sum }_{i = 1}^{{n}_{s}} {y}_{L , i , j} {M}_{i , j}}{{\sum }_{i = 1}^{{n}_{s}} {M}_{i , j}} ; and \\ {C}_{j , k} = {Cov ( {y}_{L} )}_{j , k} = \frac{{\sum }_{i = 1}^{{n}_{s}} ( {y}_{L , i , j} - {E}_{j} ) ( {y}_{L , i , k} - {E}_{k} ) {M}_{i , j} {M}_{i , k}}{( {\sum }_{i = 1}^{{n}_{s}} {M}_{i , j} {M}_{i , k} ) - 1} .$$

For the completely labeled data used by the SSVAE model, all entries corresponding to the mask matrix M are 1. Then it may be mathematically proven that the covariance matrix C is always a PSD matrix. Correspondingly, in practice, a logarithmic determinant term ln(det(C)) in the loss function value is almost always explicitly defined during the model training. However, when the entries in the mask matrix M are no longer all 1, the foregoing mathematical PSD matrix is bound to fail. Therefore, a training error may occur due to a problem of a numerical value (when the covariance matrix unexpectedly has a negative determinant, PyTorch attempts to perform a logarithmic operation on a negative number and returns a numerical error). In spite of this, since the term ln(det(C)) is merely a constant, the term may be removed from the loss function value of the ConGen model without producing any training consequence while maintaining correctness of the training.

In this embodiment of this disclosure, a real physical problem comes from quality of the covariance matrix. When availability of training data tags (namely, the molecular property tag data) is low (there are a large number of entries 0 in the mask matrix M), a major problem is to occur, because matrices E and C cannot represent a real population of molecular samples, and correspondingly, a very poor result may be produced during subsequent model training and conditional generation. If the entries of the corresponding mask matrix M are 0, the problem may be alleviated by updating the content of the tag matrix y_{L} by using the predicted data from the molecular property prediction model after each training cycle by using the interpolation technology. Then the matrices E and C are recalculated for a next training cycle, but it is assumed that all of the entries (including previously unlabeled entries) in the updated tag matrix y_{L} are valid. In other words, E and C are updated during each training period starting from an end of a first training cycle:

$${E}_{j} = {E ( y )}_{j} = \frac{{\sum }_{i = 1}^{{n}_{a}} {y}_{i , j}}{{n}_{a} - 1} ; and \\ {C}_{j , k} = {Cov ( y )}_{j , k} = \frac{{\sum }_{i = 1}^{{n}_{a}} ( {y}_{i , j} - {E}_{j} ) ( {y}_{i , k} - {E}_{k} )}{{n}_{a} - 1}$$

where the molecular property tag data respectively corresponding to the real sample population (a total sample population) is considered as y, y is continuously updated based on continuous updating of the molecular property prediction data corresponding to a sample, and n_{α} represents a quantity of all sample molecules in the real sample population. At first, the matrices E and C have very poor quality, and still cannot represent the real sample population well. However, as a predictive sub-model (i.e., a molecular property prediction network) becomes more accurate in a subsequent training iteration, E and C better represent the real sample population, so as to accordingly implement better molecular property prediction and conditional generation accuracy.

Operation 633: Adjust a parameter of the molecular generative model based on the loss function value, to obtain a trained molecular generative model.

In some embodiments, the molecular generative model includes a first generative network and a second generative network, the first generative network being configured to generate the latent space representation based on the sample molecule and the molecular property tag data of the sample molecule, the mean value and the position parameters of the probability distribution to which the latent space representation conforms are determined by parameters of the first generative network, the sample molecule, and the molecular property tag data of the sample molecule, and the second generative network being configured to obtain the generated molecule based on the latent space representation and the molecular property tag data of the sample molecule, the mean value and the position parameters of the probability distribution to which the generated molecule conforms being determined by parameters of the second generative network, the latent space representation, and the molecular property tag data of the sample molecule.

In some embodiments, the first molecular generative network is an ML network, and the second molecular generative network is also the ML network. In some embodiments, the first molecular generative network is an encoding network (encoder), and the second molecular generative network is a decoding network (decoder).

In some embodiments, after the molecular generative model is trained, a required molecular output may be conditionally generated by setting the property value of the target property and inputting a randomly sampled potential space representation (namely, the latent space representation) into the molecular generative model together. In some embodiments, a conditional property input y and the randomly sampled potential space representation z are specified to independently run the second molecular generative network, to conditionally generate the required molecular output.

In some embodiments, in some aspects of drug research, a new molecule needs to be synthesized to meet a need of drug development. Therefore, a property value or a property interval of the target property of the required generated molecule are usually given. The target property herein is determined manually, and the property value of the target property is also determined manually based on experience or a calculation result. Certainly, the molecular generative model may output a large number of generated molecules through a plurality of iterations based on the set property value or the set property interval of the target property. A distribution of the generated molecules needs to be a probability distribution that satisfies a condition of the property value or the property interval of the target property. Further, a researcher may identify one or more molecules that meet a target property condition from a large number of generated molecules.

Certainly, in addition to the drug research, virtual screening of some other materials, virtual screening of an electrolyte solution, virtual screening of catalysts, and the like may be all implemented by using the molecular generative model trained through the technical solutions provided in the Embodiments of this disclosure.

According to the technical solutions provided in the Embodiments of this disclosure, the variational encoding loss is introduced based on the mask matrix, so that the calculation of the loss of the completely labeled data and the completely unlabeled data may be combined without the need to use the molecular generative model twice. Through one training, the losses of all data in the training sample may be calculated without distinguishing between the labeled data and the unlabeled data, so as to improve the training efficiency of the model.

In addition, the accuracy of the loss function can be improved by updating a covariance matrix and a mean value of molecular tag data, so that the loss of the molecular generative model is calculated more accurately. The generation accuracy of the molecular generative model may be improved by adjusting the parameter of the molecular generative model based on the accurate loss.

In addition, the loss function is introduced to train the molecular generative model, so as to adjust the parameter of the molecular generative model. Relatively speaking, the loss function value determined based on the sample molecule and the generated molecule can better reflect the actual loss of the molecular generative model, so as to facilitate the training of the molecular generative model.

In addition, the probability distribution of the potential space representation corresponding to the training sample is obtained through the first molecular generative network. Based on the sampled potential space representation and the molecular property tag data, the probability distribution of the generated molecule is obtained through the second molecular generative network, so as to refine the specific training process of the molecular generative model and enrich the model training method. Moreover, the loss function is further represented by using a potential space representation outputted by an intermediate layer, so as to adjust the parameter of the molecular generative model, thereby further improving the generation accuracy of the molecular generative model.

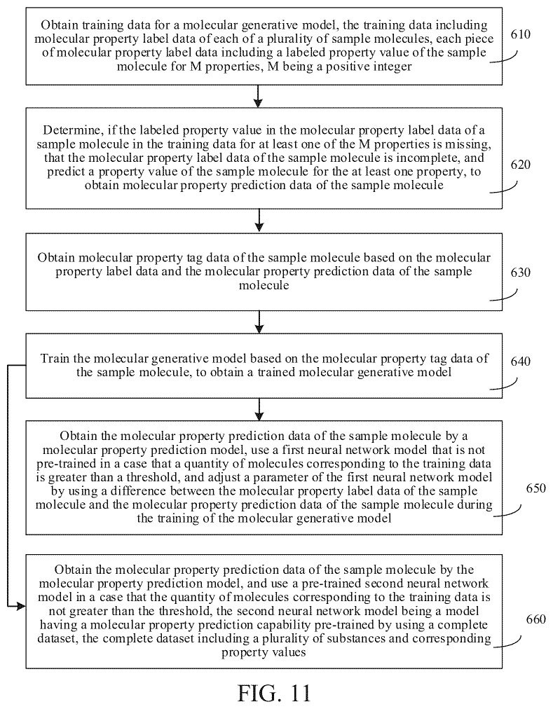

FIG. 11 is a flowchart showing a method for training a molecular generative model according to another embodiment of this disclosure. Each operation of the method may be performed by the model training device described above. In the following method embodiments, for ease of description, that each operation is performed by a “computer device” is used for description. The method may include at least one of the following operations (610-660).

Operation 610: Obtain training data for a molecular generative model, the training data including molecular property label data of each of a plurality of sample molecules, each piece of molecular property label data including a labeled property value of the sample molecule for M properties, M being a positive integer.

Operation 620: Determine, if the labeled property value in the molecular property label data of a sample molecule in the training data for at least one of the M properties is missing, that the molecular property label data of the sample molecule is incomplete, and predict a property value of the sample molecule for the at least one property, to obtain molecular property prediction data of the sample molecule.

Operation 630: Obtain molecular property tag data of the sample molecule based on the molecular property label data and the molecular property prediction data of the sample molecule.

Operation 640: Train the molecular generative model based on the molecular property tag data of the sample molecule, to obtain a trained molecular generative model.

In some embodiments, the trained molecular generative model may be configured to generate molecular data having a target property.

Operation 610 to operation 640 are the same as those described in the foregoing embodiments. For content not described in this embodiment of this disclosure, reference may be made to the foregoing embodiments. Details are not described herein again.

In some embodiments, operation 650 or operation 660 is performed after operation 640.

Operation 650: Obtain the molecular property prediction data of the sample molecule by a molecular property prediction model, use a first neural network model that is not pre-trained in a case that a quantity of molecules corresponding to the training data is greater than a threshold, and adjust a parameter of the first neural network model by using a difference between the molecular property label data of the sample molecule and the molecular property prediction data of the sample molecule during the training of the molecular generative model.

In some embodiments, the molecular property prediction model is an ML model. In some embodiments, the molecular generative model includes a molecular property prediction model, or the molecular property prediction model may also exist alone. The foregoing threshold may be set and adjusted based on an actual usage requirement, which is not limited in the Embodiments of this disclosure. The quantity of molecules corresponding to the training data refers to a total quantity of sample molecules in the training data.

In some embodiments, the first neural network model is a prediction network, which may be configured to predict a property of the sample molecule. In some embodiments, the prediction network is the foregoing predictor.

Operation 660: Obtain the molecular property prediction data of the sample molecule by the molecular property prediction model, and use a pre-trained second neural network model in a case that the quantity of molecules corresponding to the training data is not greater than the threshold, the second neural network model being a model having a molecular property prediction capability pre-trained by using a complete dataset, the complete dataset including a plurality of substances and corresponding property values.

In some embodiments, the second neural network model is the prediction network, which may also be configured to predict the property of the sample molecule. In some embodiments, the prediction network is the foregoing predictor.

In a case that the quantity of molecules corresponding to the training data is greater than the threshold, the molecular property prediction data of the molecule is predicted by using the first neural network model that is not pre-trained in this embodiment of this disclosure. This is because that a large amount of training data can provide strong sample support for the molecular property prediction model and support a subsequent property prediction task thereof. Therefore, the molecular property prediction model may be untrained, and there is no need to spend special cost to pre-train the molecular property prediction model, so as to reduce the costs required for the training.

In addition, in a case that the quantity of molecules corresponding to the training data is not greater than the threshold, the molecular property prediction data of the molecule is predicted by using the pre-trained second neural network model. This is because an insufficient amount of data in the training data cannot provide strong sample support for the molecular property prediction model and cannot support the subsequent property prediction task thereof. If the molecular property prediction model is untrained, it is likely that even after the molecular property prediction model is trained by using the training data, the prediction accuracy of the molecular property of the molecular property prediction model is still low, which cannot provide help for a subsequent molecular generative model. As a result, the training sample of the subsequent molecular generative model has insufficient accuracy, and the generated molecule has insufficient accuracy, which does not meet user needs. Therefore, in a case that the quantity of molecules corresponding to the training data is not greater than the threshold, the molecular property prediction data of the molecule is predicted by using the pre-trained second neural network model, so as to improve the accuracy of the molecular property tag data of the sample molecule, thereby improving the training accuracy of the molecular generative model. In this way, the molecule generated by using the molecular generative model has relatively high accuracy, which is more in line with a need of a developer.

In some embodiments, the second neural network model includes a backbone network and a fully connected network.

Backbone network: It is a network mainly configured to predict a molecular property. In some embodiments, the backbone network is a ChemBERTa model (a prediction network).

Fully connected network: It is a network configured to affect, by changing a parameter, molecular prediction property data outputted by the second neural network model. In some embodiments, the fully connected network is a linear layer configured to linearly adjust the output of the second neural network model.

In some embodiments, the parameter of the fully connected network may be adjusted by using the difference between the molecular property label data of the sample molecule and the molecular property prediction data of the sample molecule during the training of the molecular generative model, and a parameter of the backbone network remains unchanged.

In the technical solutions provided in the Embodiments of this disclosure, the molecular property prediction model may be flexibly selected based on an amount of data in the training sample set (namely, training data), so that the architecture of the molecular property prediction model is more flexible and changeable, so as to enrich an overall architecture of the molecular generative model and enrich the training manner of the model. In addition, the fully connected network is used to achieve the purpose of adjusting the output of the overall molecular property prediction model without changing the parameter of the backbone network, so that no inaccurate change in accuracy of the overall molecular property prediction model occurs to a great extent due to the introduction of the training sample in this application, thereby shortening a training duration of the molecular generative model and improving accuracy of an output result of the molecular generative model.

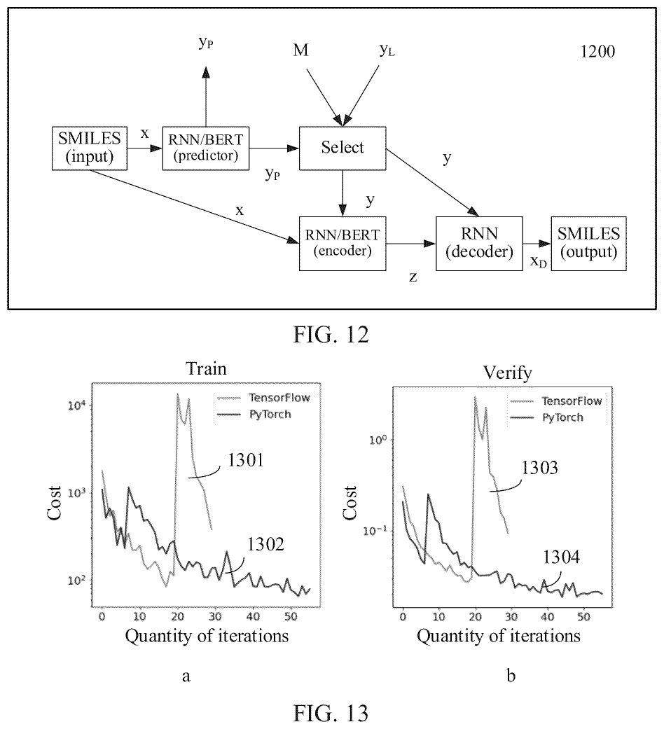

In some embodiments, a flexible sub-model (an encoder, a predictor, or the like) may be used as a substitute to enable transfer learning in the case of a very small amount of labeled data, thereby improving the training effect of the molecular generative model. Exemplarily, in many practical cases, a quantity of experimental or calculated tagged property values may be very limited. In these cases, the predictor obtained by performing simple property training on an RNN at the beginning may have a poor effect, and the RNN may be replaced with another model when needed, especially a high-precision model that has been pre-trained based on a large number of other easily available material property. A potential sub-model substitute is a ChemBERTa model, which is a large-scale pre-trained model based on a self-supervision transformer and uses the SMILES string of the molecule as an input. A fully connected network linear layer (the fully connected network) may be added on top of a transmitted ChemBERTa model (the backbone network) to obtain a new encoder. Such a transfer model (the second neural network model) is referred to as a BERT for short below. When an RNN-based encoder is replaced with the BERT, the entire ChemBERTa layer is frozen (namely, a parameter of the ChemBERTa is fixed and does not change with the training progress). However, when an RNN-based predictor is replaced with the BERT, a last layer of the ChemBERTa layer (the fully connected network) may be fine-tuned through a PyTorch optimizer. Although an RNN-based decoder is not replaced with another type of decoder sub-model, it is simple to do so in principle. 1200 of FIG. 12 is a schematic diagram of a method for training a molecular generative model and a molecular property prediction model according to an embodiment of this disclosure. The molecular property prediction model is deployed separately from the molecular generative model. The molecular property prediction model may be constructed based on an RNN or a BERT as needed, and an encoder in the molecular generative model may be constructed based on the RNN or the BERT as needed, which is not limited in this embodiment of this disclosure.

In some embodiments, training settings of the molecular generative model may be optimized. Exemplarily, to improve performance of the molecular generative model, some engineering optimization may be further performed. For the BERT model based on a pre-trained transformer, a PyTorch Adam optimizer predictor may be forced to set a BERT-based predictor sub-model to have a significantly low learning rate, for example, LR=3×10\hat{ }(−5). However, an Adam optimizer retains LR=10\hat{ }(−3) to optimize parameters of the remaining sub-models (for example, a decoder). This ensures that the BERT-based predictor sub-model may perform molecular property regression slowly and accurately without immediately corrupting a pre-training parameter of a carefully pre-trained ChemBERTa model. Compared with an SSVAE model, after a multi-conditional dirty data workflow is enabled on an RNN-based ConGen model, the models become more unstable during training, which may be alleviated by clipping a norm of a model gradient to the maximum of 10′ in a training cycle, to ensure smooth training of the RNN, thereby generating stable model training and accurate regression and conditional generation results.

In some embodiments, a code platform that carries the molecular generative model may be switched from a TensorFlow platform to a PyTorch platform. Exemplarily, the process requires a significant modification to enable the functionality of the ConGen model, which requires a more flexible Python-based ML platform. An initial SSVAE model was compiled on a TensorFlow1.0 platform, which is older and significantly less flexible than PyTorch. Therefore, a first modification to be made is to completely rewrite the SSVAE model to the PyTorch version and prove functional completeness and correctness thereof, and then make a further modification, so that the training and conditional generation capabilities of the model are roughly the same after the model is switched from the TensorFlow platform to the PyToch platform.

FIG. 13 shows comparison of performance of an SSVAE model after being switched from TensorFlow1.0 to the PyTorch platform. A result before the switching is equivalent to a result after the switching. A curve 1301 and a curve 1302 in subgraph a show a relationship between a loss and a quantity of iterations of a molecular generative model during training respectively on the TensorFlow platform 1301 and the PyTorch platform 1302. A curve 1303 and a curve 1304 in sub-graph b show a relationship between a loss and a quantity of iterations in a validation process of the molecular generative model respectively on the TensorFlow platform and the PyTorch platform. It is not difficult to find that the overall loss of the PyTorch platform is relatively low. A loss peak corresponding to the TensorFlow platform may be ignored, because it is only a result of unfortunate gradient explosion in a later period. For another example, Table 1 shows property values of a test molecule on different platforms. Table 2 shows property values of a property of a generated molecule obtained through unconditional sampling of the test molecule on different platforms. Table 3 shows property values of a property of a generated molecule obtained through conditional sampling of the test molecule on different platforms.

TABLE 1
Test molecules and molecular property values

| MAE | Mol. Wt | LogP | QED |
|---|---|---|---|
| TensorFlow | 2.48 | 0.139 | 0.0277 |
| PyTorch | 1.11 | 0.056 | 0.0108 |

TABLE 2
Unconditional sampling and molecular property values

| MAE ± Std | Mol. Wt | LogP | QED |
|---|---|---|---|
| TensorFlow | 383 ± 42 | 3.06 ± 1.27 | 0.671 ± 0.180 |
| PyTorch | 348 ± 67 | 3.09 ± 0.96 | 0.774 ± 0.119 |

TABLE 3
Conditional sampling and molecular property values

| MAE ± Std | Mol. Wt | LogP | QED |
|---|---|---|---|
| TensorFlow | 252 ± 7 | 2.00 ± 1.14 | 0.809 ± 0.073 |
| PyTorch | 250 ± 7 | 1.76 ± 0.88 | 0.793 ± 0.108 |

The main idea of the molecular generative model provided in the Embodiments of this disclosure is as follows. An architecture similar to that of the SSVAE model is used, but all components thereof are modified as needed to support the use of dirty (incomplete) training data. This effectively means that molecular generative model may be trained and used by using molecular property datasets from different sources. In practice, an example in which the molecular generative model is the ConGen model is used. A large amount of engineering and algorithm development and innovation are required to cause the ConGen model to achieve the goal.

1. Model switching from a TensorFlow platform to a PyTorch platform.
a) Engineering work is not necessary, but greatly improves flexibility of model development.

2. A training data loader is modified to allow databases to be merged and process dirty data.
a) Valid/invalid entries are labeled through an additional mask matrix including property tags to enable partial data.
b) Engineering work is crucial to implementation of the function of the ConGen model.

3. The model is modified to enable the dirty (incomplete) data workflow, instead of separating the completely labeled data from the completely unlabeled data as the SSVAE model does.
a) A new loss function is used. The loss function can automatically distinguish between labeled data and unlabeled data without needing to separate the labeled data from the unlabeled data.
b) Engineering and algorithmic work are crucial to the implementation of the function of the ConGen model.

4. Flexible sub-model replacement is performed to enable transfer learning in the case of a very small amount of labeled data.
a) Engineering work is not urgently needed, but improves the flexibility of the model.

5. A covariance matrix interpolation method is used to avoid a problem of a non-PSD covariance matrix caused by the dirty data.
a) Algorithmic work is crucial to the implementation of the function of the ConGen model.

6. Training settings of the model are optimized.
a) Selective learning rate optimization and gradient clipping are performed on a sub-model.
b) The engineering work is not urgently needed, but significantly improves model performance and consistency.

In short, the ConGen model is significantly modified compared with the SSVAE model.

In some embodiments, the training data (incomplete labeled data) is mixed from two different databases. Exemplarily, a first database includes properties such as molecular weight (MolWt), hydrophobicity (LogP), and drug-likeness (QED), and a second database includes properties such as ionization energy (IE) and electron affinity (EA). The ConGen model is trained based on all of the 5 properties.

In some embodiments, as an example of multi-property conditional generation, a generated molecule with all of the following 3 properties is generated through the molecular generative model: MolWt=250, LogP=2.5, and EA=4. Regression prediction and conditional generation results corresponding to the process are shown in Table 4, Table 5, and Table 6 below. Table 4 shows test molecules and molecular property values, Table 5 shows unconditional sampling and the molecular property values, and Table 6 shows conditional sampling and the molecular property values. It is not difficult to find that in a case that the property has a relatively large quantity of available training tags (MolWt, LogP, and QED, which may be easily obtained by using RDKit), the performance of the BERT-based ConGen model is lower than that of the RNN-based ConGen model. However, in a case that the property has a limited quantity of training tags (for example, EA and IE, which need to be calculated by using quantum chemistry software), the BERT-based ConGen model performs better processing. For the multi-property conditional sampling, it may be seen that molecules generated by the ConGen model have a required range of properties (MolWt=250, LogP=2.5), and the property values of these properties may be easily calculated and confirmed by using RDKit.

TABLE 4
Test molecules and molecular property values

| MAE | Mol. Wt | LogP | QED | EA | IE |
|---|---|---|---|---|---|
| Original RNN | 2.02 | 0.09 | 0.013 | 0.27 | 0.22 |
| Transfer BERT | 5.23 | 0.15 | 0.0174 | 0.23 | 0.17 |

TABLE 5
Unconditional sampling and molecular property values

| MAE ± Std | Mol. Wt | LogP | QED |
|---|---|---|---|
| Original RNN | 286 ± 78 | 1.93 ± 1.07 | 0.646 ± 0.161 |
| Transfer BERT | 344 ± 60 | 2.75 ± 1.11 | 0.705 ± 0.130 |

TABLE 6
Conditional sampling and molecular property values
(Mol. Wt = 250, LogP = 2.5, and EA = 4.0)

| MAE ± Std | Mol. Wt | LogP | QED |
|---|---|---|---|
| Original RNN | 239 ± 20 | 2.38 ± 0.24 | 0.641 ± 0.125 |
| Transfer BERT | 236 ± 34 | 2.42 ± 0.77 | 0.576 ± 0.139 |

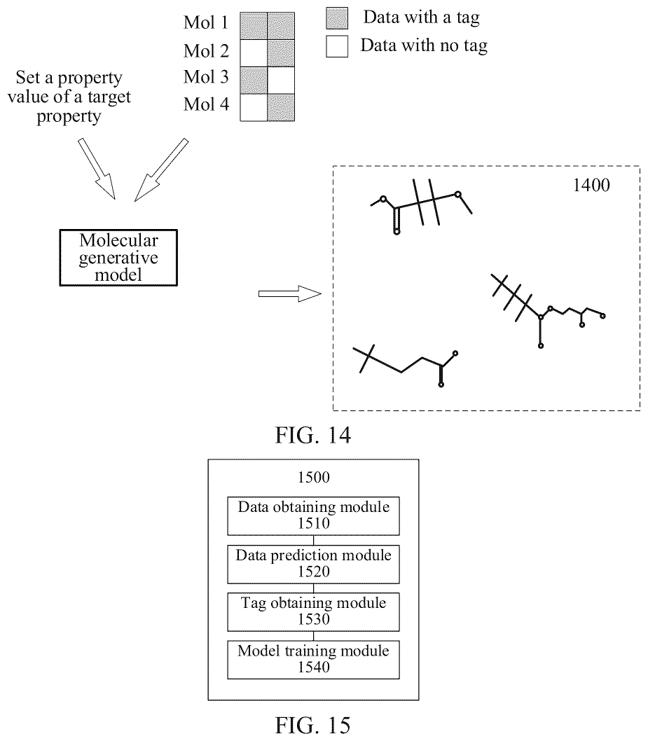

In some embodiments, FIG. 14 is an exemplary schematic diagram showing conditional molecule generation. A molecular generative model is trained by using a default database, assisted by training data (incomplete labeled data) provided by a user. After the training, a Congen model is configured to generate molecular data 1400 having multi-conditional properties of interest (i.e., property values of a set target property). The molecular data 1400 may be configured for indicating a molecular structure of a generated molecule. In some embodiments, a diluent molecule configured to automatically generate a battery electrolyte is used as an example. These diluent molecules are usually weakly polar chlorofluorocarbon-based molecules. Because these molecules are added to an actual electrolyte solution, the molecules cannot be excessively large or excessively sticky. In addition, the molecules further need to be electrochemically stable. Based on these requirements, the following target properties may be specified for the generated molecule:
1. A quantity of fluorine atoms is greater than 3;
2. A quantity of heavy atoms is less than 15;
3. Ionization energy is greater than 6.5 eV; and
4. Electron affinity is greater than 3.0 eV.

The model is first trained based on the data having the at least four target properties. Then the ConGen model is run a plurality of times (1000 times in the example above) together by using these target properties as a condition, to randomly generate 1000 related molecules. The example shown herein describes query of the battery electrolyte molecule, which may also be expanded to molecular query in other fields, for example, virtual screening of drugs and virtual screening of a cationic additive in an ammonia electrocatalytic electrolyte.

In the technical solutions provided in the Embodiments of this disclosure, a ConGen model by using a novel conditional molecule generation algorithm based on an SSVAE technology is shown. Unlike a model in the related art such as the SSVAE model, the ConGen model is carefully designed to process dirty training data with an incomplete tag. Due to various internal factors and external factors and cost considerations, a molecule that may be obtained from a publicly available database or through internal simulation and experimental measurement may have incomplete and different available property sets. The SSVAE model needs to delete a molecule with an incomplete tag or assign a virtual label to the molecule, and a trained model in this way has relatively low accuracy. However, the ConGen model may easily mix dirty training datasets from a plurality of sources to train conditional generative models thereof as needed. In addition, the ConGen model may flexibly replace a sub-model thereof with another type of model, so that a pre-trained model may be used, which is especially useful in a case that availability of the training data is limited. Based on the above, the technical solutions provided in the Embodiments of this disclosure have the following technical advantages.
1. The conditional generative model may be trained using the dirty training data. The data has incomplete property tags.
2. Incomplete training data is easily obtained by mixing molecular properties of the same type or different types from different sources, to train the ConGen model.
3. In the ConGen model, the molecules with multi-property conditions may be easily generated.
4. The sub-model in the ConGen model, especially the predictor and the encoder thereof, may be easily replaced with the pre-trained model, for example, a transformer-based BERT model (transfer learning), which is especially useful when availability of a training data tag for a specific property is relatively low.

An apparatus embodiment of this disclosure is described below, which may be configured for performing the method embodiment of this disclosure. For details not disclosed in the apparatus embodiment of this disclosure, reference is made to the method Embodiments of this disclosure.

FIG. 15 is a block showing an apparatus for training a molecular generative model according to an embodiment of this disclosure. The apparatus 1500 may include a data obtaining module 1510, a data prediction module 1520, a tag obtaining module 1530, and a model training module 1540. In this disclosure, a unit and a module may be hardware such as a combination of electronic circuitries; firmware; or software such as computer instructions. The unit and the module may also be any combination of hardware, firmware, and software. In some implementation, a unit may include at least one module. Each unit or module can be implemented using one or more processors (or processors and memory). Likewise, a processor (or processors and memory) can be used to implement one or more units or modules. Moreover, each unit or module can be part of an overall unit or module that includes the functionalities of the unit or module.

The data obtaining module 1510 is configured to obtain training data for a molecular generative model, the training data including molecular property label data of each of a plurality of sample molecules, each piece of molecular property label data including a labeled property value of the sample molecule for M properties, M being a positive integer.

The data prediction module 1520 is configured to determine, if the labeled property value in the molecular property label data of a sample molecule in the training data for at least one of the M properties is missing, that the molecular property label data of the sample molecule is incomplete, and predict a property value of the sample molecule for the at least one property, to obtain molecular property prediction data of the sample molecule.

The tag obtaining module 1530 is configured to obtain molecular property tag data of the sample molecule based on the molecular property label data and the molecular property prediction data of the sample molecule.

The model training module 1540 is configured to train the molecular generative model based on the molecular property tag data of the sample molecule, to obtain a trained molecular generative model.

In some embodiments, the tag obtaining module 1530 is configured to obtain, for each of the M properties, a predicted property value of the property from the molecular property prediction data of the sample molecule as a tagged property value of the property if the labeled property value of the property included in the molecular property label data of the sample molecule is missing.

The tag obtaining module 1530 is further configured to determine the labeled property value of the property as the tagged property value of the property if the labeled property value of the property included in the molecular property label data of the sample molecule is not missing.

The tag obtaining module 1530 is further configured to obtain the molecular property tag data of the sample molecule based on the tagged property values of the M properties.

In some embodiments, the tag obtaining module 1530 is further configured to generate a mask matrix corresponding to the training data, the mask matrix including N×M elements, each element being configured for indicating whether a labeled property value of a property of a sample molecule is missing, and N being a positive integer.

In some embodiments, the data obtaining module 1510 is configured to obtain at least two sets of completely labeled data, each set of completely labeled data including a labeled property value of at least one molecule for the at least one property, properties included in different sets of completely labeled data being different, and molecules included in the different sets of completely labeled data being different.

The data obtaining module 1510 is further configured to integrate the at least two sets of completely labeled data at a granularity of each molecule, to obtain the training data.

In some embodiments, as shown in FIG. 16, the model training module 1540 includes a molecule generating unit 1541, a loss determining unit 1542, and a model training unit 1543.

The molecule generating unit 1541 is configured to obtain a generated molecule corresponding to the sample molecule based on the sample molecule and the molecular property tag data of the sample molecule through the molecular generative model.

The loss determining unit 1542 is configured to determine a loss function value of the molecular generative model based on the sample molecule and the generated molecule, the loss function value being configured for characterizing a degree of difference between the sample molecule and the generated molecule.

The model training unit 1543 is configured to adjust a parameter of the molecular generative model based on the loss function value, to obtain the trained molecular generative model.

In some embodiments, the loss function value includes a regression loss and a variational encoding loss, the variational encoding loss including a first loss, a second loss, a third loss, and a fourth loss.

The loss determining unit 1542 is configured to determine the regression loss based on the labeled property value of the labeled property in the molecular property label data and the predicted property value of the labeled property in the molecular property prediction data, the regression loss being configured for characterizing prediction accuracy of the molecular property prediction data, and the labeled property being a property corresponding to the labeled property value that is not missing in the molecular property label data.

The loss determining unit 1542 is further configured to determine the first loss based on the generated molecule and the sample molecule, the first loss being configured for characterizing a degree of direct difference between the generated molecule and the sample molecule.

The loss determining unit 1542 is further configured to determine the second loss and the third loss based on the molecular property tag data, a covariance matrix of the molecular property tag data, a mean value of the molecular property tag data, and a mean value of the molecular property prediction data, the second loss being configured for characterizing a degree of difference between the molecular property tag data and a tagged property value of the labeled property in the molecular property tag data, and the third loss being configured for characterizing a degree of difference between the molecular property tag data and a tagged property value of another property among the M properties included in the molecular property tag data other than the labeled property.

The loss determining unit 1542 is further configured to determine the fourth loss based on a latent space representation and position parameters and a mean value of a probability distribution to which the latent space representation conforms, the fourth loss being configured for characterizing a degree of dispersion of the latent space representation relative to the probability distribution, the latent space representation being a hidden layer feature of the sample molecule obtained by an intermediate layer of the molecular generative model.

The loss determining unit 1542 is further configured to determine the variational encoding loss based on the first loss, the second loss, the third loss, and the fourth loss, the variational encoding loss being configured for characterizing a degree of difference between the sample molecule and the generated molecule generated based on the molecular property tag data.

The loss determining unit 1542 is further configured to determine the loss function value based on the regression loss and the variational encoding loss.

In some embodiments, in a case that the molecular property prediction data of the sample molecule continuously changes with the training of the molecular generative model, the covariance matrix and the mean value of the molecular property tag data are also continuously updated.

In some embodiments, the molecular generative model includes a first generative network and a second generative network, the first generative network being configured to generate the latent space representation based on the sample molecule and the molecular property tag data of the sample molecule, the mean value and the position parameters of the probability distribution to which the latent space representation conforms being determined by parameters of the first generative network, the sample molecule, and the molecular property tag data of the sample molecule, and the second generative network being configured to obtain the generated molecule based on the latent space representation and the molecular property tag data of the sample molecule, the mean value and the position parameters of the probability distribution to which the generated molecule conforms being determined by parameters of the second generative network, the latent space representation, and the molecular property tag data of the sample molecule.

In some embodiments, the molecular property prediction data of the sample molecule is obtained by a molecular property prediction model.

The model training module 1540 is further configured to use a first neural network model that is not pre-trained in a case that a quantity of molecules corresponding to the training data is greater than a threshold, and adjust a parameter of the first neural network model by using a difference between the molecular property label data of the sample molecule and the molecular property prediction data of the sample molecule during the training of the molecular generative model; or use a pre-trained second neural network model in a case that the quantity of molecules corresponding to the training data is not greater than the threshold, the second neural network model being a model having a molecular property prediction capability pre-trained by using a complete dataset, the complete dataset including a plurality of substances and corresponding property values.

In some embodiments, the second neural network model includes a backbone network and a fully connected network,
a parameter of the fully connected network being adjusted by using the difference between the molecular property label data of the sample molecule and the molecular property prediction data of the sample molecule during the training of the molecular generative model, and a parameter of the backbone network remaining unchanged.

According to the technical solutions provided in the Embodiments of this disclosure, the molecular generative model is trained by using the training data with a missing labeled property value, so that newly generated molecular data has more abundant molecular properties that are not limited to a few molecular properties specified in complete labeled data, thereby improving diversity of the generated molecular data. In addition, the training data may include a plurality of pieces of complete labeled data. Therefore, types of the molecular properties of the sample molecule can be enriched through the training data, thereby further improving diversity of output results of the molecular generative model.

In addition, since the labeled property values for the M properties in the training data are missing, a data gap in incomplete labeled data is filled in by obtaining the molecular property prediction data corresponding to the sample molecule, so as to improve completeness of the training sample, achieve a smoother subsequent training process, and facilitate improvement in training accuracy of the molecular generative model.

In addition, different from a training manner in which the molecular generative model is trained by using only the complete labeled data in the related art, in the technical solutions provided in the Embodiments of this disclosure, the training data with a missing labeled property value may also be configured for training the molecular generative model. Therefore, the method for training a molecular generative model is also enriched.

When the apparatus provided in the foregoing embodiment implements the functions of the apparatus, only division of the foregoing functional modules is used as an example for description. In a practical application, the functions may be completed by different functional modules as required. To be specific, an internal structure of a device is divided into different functional modules to complete all or part of the functions described above. In addition, the apparatus provided in the foregoing embodiment belongs to the same idea as the method embodiment. For details of the specific implementation process thereof, reference is made to the method embodiment. Details are not described herein again.

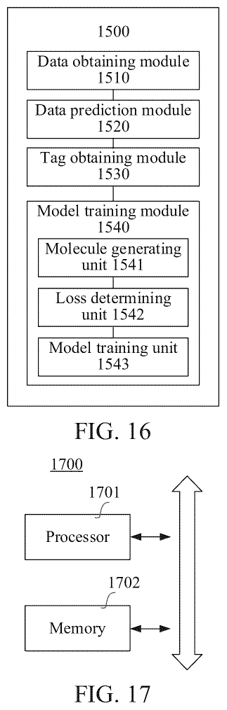

FIG. 17 is a schematic structural diagram of a computer device according to an exemplary embodiment of this disclosure.

Generally, the computer device 1700 includes a processor 1701 and a memory 1702.

The processor 1701 may include one or more processing cores, for example, a 4-core processor or a 17-core processor. The processor 1701 may be implemented by using at least one of the following hardware forms: digital signal processing (DSP), a field-programmable gate array (FPGA), and a programmable logic array (PLA). The processor 1701 may also include a main processor and a coprocessor. The main processor is a processor configured to process data in an awake state, and is also referred to as a central processing unit (CPU). The coprocessor is a low-power processor configured to process data in a standby state. In some embodiments, the processor 1701 may be integrated with a graphics processing unit (GPU). The GPU is configured to render and draw content that needs to be displayed on a display screen. In some embodiments, the processor 1701 may further include an AI processor. The AI processor is configured to process computing operations related to machine learning.

The memory 1702 may include one or more non-transitory computer-readable storage media. The non-transitory computer-readable storage medium may be tangible and non-transient. The memory 1702 may further include a high-speed random access memory (RAM) and a nonvolatile memory, for example, one or more disk storage devices or flash storage devices. In some embodiments, the non-transient computer-readable storage medium in the memory 1702 has a computer program stored therein. The computer program is loaded and executed by the processor 1701 to implement the method for training a molecular generative model provided in the foregoing method embodiments.

A person skilled in the art may understand that the structure shown in FIG. 17 does not constitute a limitation on the computer device 1700, and the computer device may include more or fewer components than those shown in the figure, or some merged components, or different component arrangements.

In an exemplary embodiment, a non-transitory computer-readable storage medium is further provided, having a computer program stored therein, the computer program, when executed by a processor, implementing the method for training a molecular generative model.

In some embodiments, the non-transitory computer-readable storage medium may include a read-only memory (ROM), a RAM, a solid state drive (SSD), an optical disc, or the like. The RAM may include a resistance RAM (ReRAM) and a dynamic RAM (DRAM).

In an exemplary embodiment, a computer program product is further provided. The computer program product includes a computer program, the computer program being stored in a non-transitory computer-readable storage medium. The processor of the computer device reads the computer program from the non-transitory computer-readable storage medium. The processor executes the computer program, so that the computer device performs the foregoing method for training a molecular generative model.

According to the Embodiments of this disclosure, a prompt interface and a pop-up window may be displayed or voice prompt information is outputted before relevant data of a user is collected and during collection of relevant data of the user. The prompt interface, the pop-up window, or the voice prompt information is configured for prompting that relevant data of the user is being collected currently, so that this application starts the relevant operations of obtaining user-related data only after obtaining a confirm operation performed by the user on the prompt interface or the pop-up window, or otherwise (i.e., when the confirm operation performed by the user on the prompt interface or the pop-up window is not obtained), the relevant operations of obtaining user-related data are ended, i.e., the user-related data is not obtained. In other words, all user data collected in this application is processed in strict accordance with requirements of relevant national laws and regulations, and informed consent or individual consent of a subject of personal information is collected with consent and authorization of the user. Within the scope of authorization of laws and regulations and the subject of personal information, subsequent use and processing behaviors of data are carried out, and the collection, use, and processing of relevant user data need to comply with the relevant laws, regulations, and standards of relevant countries and regions. For example, the incomplete labeled data and the sample molecule involved in this application are obtained with full authorization.

“Plurality of” mentioned in the specification means two or more. The term “and/or” is an association relationship for describing associated objects and represents that three relationships may exist. For example, A and/or B may represent the following three cases: only A exists, both A and B exist, and only B exists. The character “/” generally indicates an “or” relationship between a preceding associated object and a latter associated object. In addition, the operation numbers described in this specification merely exemplarily show a possible execution sequence of the operations. In some other embodiments, the foregoing operations may not be performed based on the number sequence. For example, two operations with different numbers may be performed simultaneously, or two operations with different numbers may be performed based on a sequence contrary to the sequence shown in the figure. This is not limited in the Embodiments of this disclosure.

The foregoing descriptions are merely exemplary Embodiments of this disclosure, and are not intended to limit this application. Any modification, equivalent replacement, or improvement made within the spirit and principle of this application falls within the protection scope of this application.

What is claimed is:

1. A method performed by a computer device, comprising:
obtaining training data for a molecular generative model, the training data comprising molecular property label data for a plurality of sample molecules, each of the plurality of sample molecules having M properties, each piece of molecular property label data being corresponding to a sample molecule in the plurality of sample molecules and comprising labeled property values, each of the labeled property values applying to one of the M properties, M being a positive integer;
determining, if there is no labeled property value corresponding to a first property in the M properties of the sample molecule, that the molecular property label data is incomplete, and predicting a predicted property value of the first property, to obtain molecular property prediction data of the sample molecule;
obtaining molecular property tag data of the sample molecule based on the molecular property label data and the molecular property prediction data of the sample molecule; and
training the molecular generative model based on the molecular property tag data of the sample molecule, to obtain a trained molecular generative model.

2. The method according to claim 1, wherein obtaining the molecular property tag data of the sample molecule comprises:
obtaining, a predicted property value of the first property from the molecular property prediction data of the sample molecule as a first tagged property value of the first property;
determining a labeled property value of a second property in the M properties of the sample molecule as a second tagged property value of the second property if the labeled property value of the second property comprised in the molecular property label data of the sample molecule is not missing; and
obtaining the molecular property tag data of the sample molecule based on the tagged property values of the M properties.

3. The method according to claim 1, further comprising:
generating a mask matrix corresponding to the training data, the mask matrix comprising N×M elements, each element being configured for indicating whether a labeled property value of a property of a sample molecule is missing, and N being a positive integer.

4. The method according to claim 1, wherein obtaining training data for the molecular generative model comprises:
obtaining at least two sets of completely labeled data, each set of completely labeled data comprising a labeled property value for one property of a molecule, property values comprised in different sets of completely labeled data being different, and molecules comprised in the different sets of completely labeled data being different; and
integrating the at least two sets of completely labeled data at a granularity of molecule level, to obtain the training data.

5. The method according to claim 1, wherein training the molecular generative model comprises:
obtaining a generated molecule corresponding to the sample molecule based on the sample molecule and the molecular property tag data of the sample molecule through the molecular generative model;
determining a loss function value of the molecular generative model based on the sample molecule and the generated molecule, the loss function value being configured for characterizing a degree of difference between the sample molecule and the generated molecule; and
adjusting a parameter of the molecular generative model based on the loss function value, to obtain the trained molecular generative model.

6. The method according to claim 5, wherein:
the loss function value comprises a regression loss and a variational encoding loss;
the variational encoding loss comprises a first loss, a second loss, a third loss, and a fourth loss; and
determining the loss function value of the molecular generative model comprises:
determining the regression loss based on the labeled property values of the labeled property in the molecular property label data and the predicted property value, the regression loss being configured for characterizing prediction accuracy of the molecular property prediction data, and the labeled property being a property corresponding to the labeled property value that is not missing in the molecular property label data;
determining the first loss based on the generated molecule and the sample molecule, the first loss being configured for characterizing a degree of direct difference between the generated molecule and the sample molecule;
determining the second loss and the third loss based on the molecular property tag data, a covariance matrix of the molecular property tag data, a mean value of the molecular property tag data, and a mean value of the molecular property prediction data, the second loss being configured for characterizing a degree of difference between the molecular property tag data and a tagged property value of the labeled property in the molecular property tag data, and the third loss being configured for characterizing a degree of difference between the molecular property tag data and a tagged property value of another property among the M properties comprised in the molecular property tag data other than the labeled property;
determining the fourth loss based on a latent space representation and position parameters and a mean value of a probability distribution to which the latent space representation conforms, the fourth loss being configured for characterizing a degree of dispersion of the latent space representation relative to the probability distribution, the latent space representation being a hidden layer feature of the sample molecule obtained by an intermediate layer of the molecular generative model;
determining the variational encoding loss based on the first loss, the second loss, the third loss, and the fourth loss, the variational encoding loss being configured for characterizing a degree of difference between the sample molecule and the generated molecule generated based on the molecular property tag data; and
determining the loss function value based on the regression loss and the variational encoding loss.

7. The method according to claim 6, wherein in response to the molecular property prediction data of the sample molecule changing with the training of the molecular generative model, updating the covariance matrix and the mean value of the molecular property tag data.

8. The method according to claim 6, wherein:
the molecular generative model comprises a first generative network and a second generative network;
the first generative network being configured to generate the latent space representation based on the sample molecule and the molecular property tag data of the sample molecule, the mean value and the position parameters of the probability distribution to which the latent space representation conforms being determined by parameters of the first generative network, the sample molecule, and the molecular property tag data of the sample molecule; and
the second generative network being configured to obtain the generated molecule based on the latent space representation and the molecular property tag data of the sample molecule, the mean value and the position parameters of the probability distribution to which the generated molecule conforms being determined by parameters of the second generative network, the latent space representation, and the molecular property tag data of the sample molecule.

9. The method according to claim 1, wherein:
the molecular property prediction data of the sample molecule is obtained by a molecular property prediction model;
in response to a quantity of molecules corresponding to the training data is greater than a threshold, the molecular property prediction model is implemented as a first neural network model that is not pre-trained, and a parameter of the first neural network model is adjusted by using a difference between the molecular property label data of the sample molecule and the molecular property prediction data of the sample molecule during the training of the molecular generative model; and
in response to a quantity of molecules corresponding to the training data is greater than a threshold, the molecular property prediction model is implemented as a second neural network model that is pre-trained, the second neural network model being a model having a molecular property prediction capability pre-trained by using a complete dataset, the complete dataset comprising a plurality of substances and corresponding property values.

10. The method according to claim 9, wherein: the second neural network model comprises a backbone network and a fully connected network,
the second neural network model comprises a backbone network and a fully connected network; and
a parameter of the fully connected network being adjusted by using the difference between the molecular property label data of the sample molecule and the molecular property prediction data of the sample molecule during the training of the molecular generative model, and a parameter of the backbone network remaining unchanged.

11. A device comprising a memory for storing computer instructions and a processor in communication with the memory, wherein, when the processor executes the computer instructions, the processor is configured to cause the device to:
obtain training data for a molecular generative model, the training data comprising molecular property label data for a plurality of sample molecules, each of the plurality of sample molecules having M properties, each piece of molecular property label data being corresponding to a sample molecule in the plurality of sample molecules and comprising labeled property values, each of the labeled property values applying to one of the M properties, M being a positive integer;
determine, if there is no labeled property value corresponding to a first property in the M properties of the sample molecule, that the molecular property label data is incomplete, and predict a predicted property value of the first property, to obtain molecular property prediction data of the sample molecule;
obtain molecular property tag data of the sample molecule based on the molecular property label data and the molecular property prediction data of the sample molecule; and
train the molecular generative model based on the molecular property tag data of the sample molecule, to obtain a trained molecular generative model.

12. The device according to claim 11, wherein, when the processor is configured to cause the device to obtain the molecular property tag data of the sample molecule, the processor is configured to cause the device to:
obtain, a predicted property value of the first property from the molecular property prediction data of the sample molecule as a first tagged property value of the first property;
determine a labeled property value of a second property in the M properties of the sample molecule as a second tagged property value of the second property if the labeled property value of the second property comprised in the molecular property label data of the sample molecule is not missing; and
obtain the molecular property tag data of the sample molecule based on the tagged property values of the M properties.

13. The device according to claim 11, wherein, when the processor executes the computer instructions, the processor is configured to further cause the device to:
generate a mask matrix corresponding to the training data, the mask matrix comprising N×M elements, each element being configured for indicating whether a labeled property value of a property of a sample molecule is missing, and N being a positive integer.

14. The device according to claim 11, wherein, when the processor is configured to cause the device to obtain training data for the molecular generative model, the processor is configured to cause the device to:
obtain at least two sets of completely labeled data, each set of completely labeled data comprising a labeled property value for one property of a molecule, property values comprised in different sets of completely labeled data being different, and molecules comprised in the different sets of completely labeled data being different; and
integrate the at least two sets of completely labeled data at a granularity of molecule level, to obtain the training data.

15. The device according to claim 11, wherein, when the processor is configured to cause the device to train the molecular generative model, the processor is configured to cause the device to:
obtain a generated molecule corresponding to the sample molecule based on the sample molecule and the molecular property tag data of the sample molecule through the molecular generative model;
determine a loss function value of the molecular generative model based on the sample molecule and the generated molecule, the loss function value being configured for characterizing a degree of difference between the sample molecule and the generated molecule; and
adjust a parameter of the molecular generative model based on the loss function value, to obtain the trained molecular generative model.

16. The device according to claim 15, wherein:
the loss function value comprises a regression loss and a variational encoding loss;
the variational encoding loss comprises a first loss, a second loss, a third loss, and a fourth loss; and
when the processor is configured to cause the device to determine the loss function value of the molecular generative model comprises:
determine the regression loss based on the labeled property values of the labeled property in the molecular property label data and the predicted property value, the regression loss being configured for characterizing prediction accuracy of the molecular property prediction data, and the labeled property being a property corresponding to the labeled property value that is not missing in the molecular property label data;
determine the first loss based on the generated molecule and the sample molecule, the first loss being configured for characterizing a degree of direct difference between the generated molecule and the sample molecule;
determine the second loss and the third loss based on the molecular property tag data, a covariance matrix of the molecular property tag data, a mean value of the molecular property tag data, and a mean value of the molecular property prediction data, the second loss being configured for characterizing a degree of difference between the molecular property tag data and a tagged property value of the labeled property in the molecular property tag data, and the third loss being configured for characterizing a degree of difference between the molecular property tag data and a tagged property value of another property among the M properties comprised in the molecular property tag data other than the labeled property;
determine the fourth loss based on a latent space representation and position parameters and a mean value of a probability distribution to which the latent space representation conforms, the fourth loss being configured for characterizing a degree of dispersion of the latent space representation relative to the probability distribution, the latent space representation being a hidden layer feature of the sample molecule obtained by an intermediate layer of the molecular generative model;
determine the variational encoding loss based on the first loss, the second loss, the third loss, and the fourth loss, the variational encoding loss being configured for characterizing a degree of difference between the sample molecule and the generated molecule generated based on the molecular property tag data; and
determine the loss function value based on the regression loss and the variational encoding loss.

17. The device according to claim 16, wherein, when the processor executes the computer instructions, the processor is configured to further cause the device to:
in response to the molecular property prediction data of the sample molecule changing with the training of the molecular generative model, update the covariance matrix and the mean value of the molecular property tag data.

18. The device according to claim 16, wherein:
the molecular generative model comprises a first generative network and a second generative network;
the first generative network being configured to generate the latent space representation based on the sample molecule and the molecular property tag data of the sample molecule, the mean value and the position parameters of the probability distribution to which the latent space representation conforms being determined by parameters of the first generative network, the sample molecule, and the molecular property tag data of the sample molecule; and
the second generative network being configured to obtain the generated molecule based on the latent space representation and the molecular property tag data of the sample molecule, the mean value and the position parameters of the probability distribution to which the generated molecule conforms being determined by parameters of the second generative network, the latent space representation, and the molecular property tag data of the sample molecule.

19. A non-transitory storage medium for storing computer readable instructions, the computer readable instructions, when executed by a processor, causing the processor to:
obtain training data for a molecular generative model, the training data comprising molecular property label data for a plurality of sample molecules, each of the plurality of sample molecules having M properties, each piece of molecular property label data being corresponding to a sample molecule in the plurality of sample molecules and comprising labeled property values, each of the labeled property values applying to one of the M properties, M being a positive integer;
determine, if there is no labeled property value corresponding to a first property in the M properties of the sample molecule, that the molecular property label data is incomplete, and predict a predicted property value of the first property, to obtain molecular property prediction data of the sample molecule;
obtain molecular property tag data of the sample molecule based on the molecular property label data and the molecular property prediction data of the sample molecule; and
train the molecular generative model based on the molecular property tag data of the sample molecule, to obtain a trained molecular generative model.

20. The non-transitory storage medium according to claim 11, wherein, when the computer readable instructions cause the processor to obtain the molecular property tag data of the sample molecule, the computer readable instructions cause the processor to:
obtain, a predicted property value of the first property from the molecular property prediction data of the sample molecule as a first tagged property value of the first property;
determine a labeled property value of a second property in the M properties of the sample molecule as a second tagged property value of the second property if the labeled property value of the second property comprised in the molecular property label data of the sample molecule is not missing; and
obtain the molecular property tag data of the sample molecule based on the tagged property values of the M properties.
